(19) United States
(12) Patent Application Publication
(10) Pub. No.: US 2023/0142647 A1
(43) Pub. Date: May 11, 2023

(54) METHOD OF TREATING CANCER OR A BLOOD DISORDER

(71) Applicant: Quteba EBRAHEM, North Olmsted, OH (US)

(72) Inventor: Quteba EBRAHEM, North Olmsted, OH (US)

(21) Appl. No.: 17/862,879

(22) Filed: Jul. 12, 2022

Related U.S. Application Data
(60) Provisional application No. 63/220,809, filed on Jul. 12, 2021.

(51) Int. Cl. A61K 31/7072; A61K 31/706; A61K 31/352; A61K 31/216; A61K 45/06; A61P 35/02; A61P 7/00; A61K 31/7068; A61K 31/497

(52) U.S. Cl. CPC A61K 31/7072; A61K 31/706; A61K 31/352; A61K 31/216; A61K 45/06; A61P 35/02; A61P 7/00; A61K 31/7068; A61K 31/497

(57) ABSTRACT

This disclosure provides a method of treating cancer or a blood disorder disease in a patient, the method comprising administering a therapeutically effective amount of a combination of compounds comprising (1) a hypomethylating agent (HMA) and (2) an XPO1 inhibitor to a patient. The XPO1 inhibitor can be Valtrate or Caffeic acid phenyl ester (CAPE) or salt, hydrate or derivative thereof. Cancers that may be treated with this method include acute myeloid leukemia (AML) particularly leukemias in which a MLL mutation is present or one or both of an NPM1 mutation and an FLT3 mutation is present.

### CROSS REFERENCE TO RELATED APPLICATION

This application claims priority to U.S. Provisional Appl. No. 63/220,809, filed Jul. 12, 2021, which is hereby incorporated by reference in its entirety.

### BACKGROUND

Acute myeloid leukemia (AML) is the most common types of leukemia in adults, accounting for 80% of leukemia cases. It is characterized by uncontrolled growth of undifferentiated myeloid precursors i.e. myeloblasts in the bone marrow, leading to their accumulation in the bone marrow and eventually peripheral blood. These cells fail to undergo normal hematopoiesis resulting in reduced production of neutrophils, red blood cells and platelets.

AML is a highly heterogeneous disease with multiple molecular and cytogenetic features, clinical representations, therapy outcomes, and survival rates. Typical treatment options in patients with normal- and favorable-risk-cytogenetics, such as Inversion (16) or Translocation (8;21) in the absence of stem cell-like genes and nucleophosmin 1 (NPM1) mutations, are remission-inducing intensive chemotherapy, cytarabine and an anthracycline drug, followed by consolidation chemotherapy. In patients with an intermediate- or high-risk relapse, such as those with FLT3 mutations, monosomy karyotypes, intermediate-, high-risk-, or complex-cytogenetics, or who failed to achieve remission with the initial induction therapy, the treatment choice is hematopoietic stem cell transplantation (HSCT). With this standard therapeutic regimen, 70-80% of AML patients <60 years age show good overall response and a complete remission, however, most of these patients will suffer relapse. In addition, poor response rates have been observed for the conventional treatment in patients with high-risk mutations, such as TP53 [1, 2]. This, in part, can be attributed to the molecular and genetic heterogeneity of the disease that affects disease progression and, therefore, is a major obstacle in prognosis classification and clinical management of the patients. Moreover, intensive chemotherapy is not the treatment choice for patients >60 years of age due to toxicity issues; these patients have a median survival of only 5-10 months. Thus, disease relapse, treatment resistance, and therapy-induced mortality are significant challenges, and as many as 80% of older patients and 50% of patients <60 years age succumb to the disease. Taken together, the long-term outcomes of current treatment strategy in AML are poor, and complete remission is generally insufficient to improve overall survival.

Although longer treatments have been possible with the introduction of small molecule inhibitors, such as imatinib and dasatinib, and monoclonal antibodies, such as rituximab, these treatments are associated with serious grade 3 and 4 toxicities. A single-institution analysis for percentage improvement in AML 5-year overall survival (OS) over a period of 16 years demonstrated mere 19% to 35% improvement for patients under 60 and <11% in patients older than 60. There remains a high-unmet need for better AML treatments that improve survival and quality of life for AML patients.

In eukaryotic cells, proper spatiotemporal localization of biomolecules within nucleus and cytoplasmic compartments is crucial for the cells' physiological functioning and to sustain cell survival and development. Transport of small molecules across the nuclear membrane may occur by passive diffusion through the nuclear pore complex (NPC) or via vesicles derived from nuclear envelope budding. However, for larger (>40 kDa) molecules, including the vast majority of proteins, spatial compartmentalization is regulated by the process of nucleocytoplasmic shuttling, an energy-dependent, selective, and efficient system for trafficking proteins and other macromolecules across the nuclear envelope through the NPC. Aberrant nucleocytoplasmic shuttling may affect important cellular processes such as cellular growth, inflammatory response, cell cycle, and apoptosis.

Imbalance of nucleocytoplasmic shuttling frequently occurs in cancers and is emerging as a cancer hallmark. Mis-localization of tumor suppressors and oncoproteins enhances tumorigenesis and allows cancerous cells to evade terminal differentiation. In several cancers major tumor suppressors become functionally inactivated due to aberrant subcellular localization. In healthy cells tumor suppressor proteins (TSPs) that prevent cancer initiation and progression and promote the response to chemotherapy are normally localized in the nucleus and transcribe specific genes that regulate cell cycle and cell proliferation. In cancer cells these tumor suppressor proteins are frequently exported from the nucleus to the cytoplasm and disabled. These tumor suppressor genes include P53, p21, P27, FOXO, RUNX3, APC, NPM1, and Fbw7γ (Table 1). Additionally, cancer cells also shift oncogenic proteins from the cytoplasm to nucleus to maximize their transcriptional activity, further enhancing carcinogenesis. Some of these oncogenes are β-Catenin, NF-kB, BRAC1 and HIF-1α (Table 1). Importantly, increased expression of karyopherins in cancer cells is a common finding, which suggests that cancer cells may be dependent on the nuclear transport molecular machinery for their growth and survival. Anti-cancer therapies that selectively target nuclear transport machinery in cancer cells are needed due to the dependence of cancer cells on altered nucleo-cytoplasmic levels of essential proteins.

TABLE 1
Sub cellular localization of tumor suppressor and
oncogenic proteins in normal and cancer cells.

| | Molecular | Localization in | Localization in | |
|---|---|---|---|---|
| Protein | function | normal cells | cancer cells | Reference |
| P53 | Transcription factor | Nucleus | Cytoplasm | [3] |
| P27 | Cell cycle inhibitor | Nucleus | Cytoplasm | |
| Foxo | Transcription factor | Nucleus | Cytoplasm | |
| RUNX3 | Transcription factor | Nucleus | Cytoplasm | |
| NPM1 | Ribonucleoprotein | Nucleus | Cytoplasm | |
| Fbw7γ | Ubiquitin ligase | Nucleus | Cytoplasm | [4] |
| P21 | Cell cycle inhibitor | Nucleus | Cytoplasm | |
| β-Catenin | Wnt signaling | Cytoplasm | Nucleus | |
| NF-kB | Transcription factor | Cytoplasm | Nucleus | [5] |
| HIF-1α | Transcription factor | Cytoplasm | Nucleus | [6] |
| BRCA1 | DNA repair | Cytoplasm | Nucleus | [7] |

Nucleocytoplasmic transport is an active process that requires a specialized transport system comprising 3 components, NPCs, karyopherins, and RAN GTPase (Ras-related nuclear GTPase). NPCs are proteinaceous, aqueous channels that perforate the nuclear membrane. Karyopherins are a family of soluble transport receptors which recognize specific amino acid sequences their cargo proteins. These sequences are the nuclear localization signal (NLS, basic-residue rich) and the nuclear export signal (NES, leucine-rich). Transport of a majority of proteins across the NPC is mediated by the karyopherin-β family, which is further classified into importins (import target cargo into the nucleus) and exportins (export target cargo out of the nucleus) or biportins (transport target cargo in both directions). RAN GTPase cycles between GTP (RAN-GTP) and GDP (RAN-GDP)-bound forms. The guanine nucleotide exchange factor regulator of chromosome condensation 1 (RCC1) for RAN is tethered to the chromatin, therefore, RAN occurs in GTP-bound state in the nucleus. However, RAN GTPase-activating protein (RANGAP) and RAN-binding protein (RANBP1) that activate GTP hydrolysis reside in the cytoplasm. Therefore, RAN occurs in GDP-bound state in the cytoplasm. The RAN-GTP/RAN-GDP gradient regulates spatial-directionality of the nucleocytoplasmic transport. Exportins will only bind their NES-containing cargos where RAN-GTP is present i.e. in the nucleus. The formation of ternary complex, exportin-RanGTP-cargo in the nucleus begins the nuclear export process. Once in the cytoplasm, RanGTP encounters RANGAP1 and RANBP1 that hydrolyze RAN-GTP to RAN-GDP. This results in the disruption of the ternary complex and release of cargo into cytoplasm. The opposite process occurs in the nuclear import process. Importins bind their cargos with positive cooperativity in the presence of RAN-GDP only in the cytoplasm. The formation of the importin-RAN-GDP-cargo complex powers nuclear importation. In the nucleus RAN-GTP replaces RAN-GDP due to its high affinity towards importins, resulting in cargo release.

Among karyopherins, exportins are a major research focus as potential targets in tumorigenesis. Exportin1 or XPO1, the first exportin to be discovered, is also the most well-characterized exportin [8, 9]. In almost 20 years of research, over 200 NES-containing, bona-fide cargos of XPO1 have been identified, including multiple tumor suppressor proteins and oncoproteins. A study using tandem mass spectrometry analysis has further expanded the cargo-spectrum of XPO1 and identified more than 1000 cellular proteins in the XPO1-dependent nuclear exportome. The human XPO1 gene is located on chromosome 2p15. Human XPO1 is a 120-kDa protein, organized into a ring- or toroidal-shaped, inner concave and outer convex, structure made up of 21 HEAT repeats and a C-terminal helix. Leucine-rich NES-containing cargo binds to a hydrophobic groove formed by HEAT repeats 11 and 12 at the exterior convex surface of XPO1. RAN-GTP binds to the inner surface of XPO1. In the absence of RAN-GTP, HEAT repeat 9 and the C-helix bind to the inner side of the HEAT repeats 11 and 12, giving rise to a low affinity-conformation to the NES-binding groove, therefore, XPO1 binds to the NES of its cargo with a low affinity. Binding of RAN-GTP strengthens this interaction by allosterically rearranging HEAT repeats 11 and 12 giving rise to a high-affinity conformation. In the cytoplasm RAN-GTP is hydrolyzed to RAN-GDP by RANGAP1 and RANBP1, causing a movement of HEAT repeat 9 and the C-helix, and leading to the rotation of HEAT repeats −11 and −12, followed by cargo release. Indeed, mutations such as E571 within the NES-binding groove greatly reduce the affinity of XPO1 to its cargo, and mutations within the HEAT repeat 9 or the deletion of the C-helix enhance the affinity of XPO1 to its cargo and reduce the rate of cargo release.

Many cancers including leukemia, and solid cancers, display high XPO1 mRNA and protein levels of XPO1. High XPO1 expression correlates with shorter OS and adverse-risk AML. Increased XPO1 levels were significantly associated with low median OS (37 weeks) compared to low XPO1 levels (66 weeks, p=0.007) in a multivariate analysis. Increased expression of XPO1, in a reverse phase protein array analysis in AML patients (n=511), correlated with clinical parameters of poor prognosis, including higher white blood cell- and absolute peripheral blood blast-counts, and higher blast percentages in bone marrow and peripheral blood. Although the exact molecular mechanisms underlying CRM1/XPO1 overexpression remain unknown, XPO1 gene amplification or copy number gain (locus 2p16.1-2p15) has been reported in multiple hematological malignancies. Moreover, Glu571 to Lys/Gly (E571K/G), Asp724 (D724), and Arg749 (R749) missense mutations in the hydrophobic grove are a recurrent phenomenon in multiple solid and hematologic cancers [5, 6]. These mutations possibly disrupt the open-closed state equilibrium, and shape of XPO1 leading to differential cargo specificity and binding affinities.

Cancer cells utilize the XPO1 protein to mislocalize tumor suppressors and oncogenic proteins. Preclinical studies have demonstrated a role of XPO1-mediated transport in regulating p53 pathway activation in TP53 mutated AML. Moreover, increased expression of XPO1 is associated with high-risk, FMS-like tyrosine kinase 3 (FLT3), mutations in AML. And, a synergism was observed between XPO1- and FLT3-inhibitors in inducing apoptosis in preclinical studies in AML. This synergism was attributed to retention of tumor suppressors in the nucleus. XPO1 function is not just limited to the transport of TSP cargoes but also has a role in drug resistance, retaining master transcription factors essential for cell differentiation, cell survival, and autophagy. NPM1 mutations, found in one-third of AML patients, that substitute an NLS within NES as a result of frameshift in the amino acid sequence, leads to aberrant accumulation of NPM1 in the cytoplasm through interaction with XPO1. Aberrant concentration of NPM1 in the cytoplasm in AML co-translocates and dislocates the master regulator PU.1 from its interaction with CEBPA/RUNX1 transcription factor complex, and this disrupts monocyte differentiation. Another major consequence associated with abnormal cytoplasmic localization of NPM1 is upregulation of homeobox (HOX) genes and their cofactors MEIS1 and PBX3. An overexpression of HOX/MEIS1/PBX3 transcription program is responsible for maintaining leukemic cells in undifferentiated state in AML. Further, XPO1-dependent nucleo-cytoplasmic shuttling of BCL-2 and MCL-1 has been shown to regulate translation of these anti-apoptotic proteins. XPO1 is also an important player in regulating the localization of mitotic proteins to specific regions of the mitotic spindle as well as in stabilizing the kinetochore to ensure proper chromosomal segregation. Thus, XPO1 regulates mitosis in addition to its role in nuclear transport. Taken together, XPO1 is a remarkable prognostic marker and an attractive therapeutic target for restoring normal localization and function of tumor suppressors and oncoproteins.

The role of nucleocytoplasmic shuttling in cancer is well recognized, and there is rapidly growing interest in targeting the nuclear export system using small molecule inhibitors to relieve the abnormal nucleo-cytoplasmic cargo imbalance in cancer cells. The importance of this process in cancers can be realized from the accelerated FDA approvals granted to selinexor (XPOVIO) (an XPO1 inhibitor) in multiple myeloma and diffused large cell B cell lymphoma in 2020 despite the serious side effects associated with the drug. Unfortunately, despite promising preclinical data, XPO1 inhibitors have failed to show such promise in the clinic in AML. Thus, new methods for increasing the safety profile of XPO1 inhibitors by improvised strategies to screen small molecule inhibitors targeting XPO1, and/or drug combinations encompassing XPO1 inhibitors at a reduced dosage has the potential to successfully translate XPO1 inhibitors to the clinic in AML.

Epigenetic alterations are inherited modifications in DNA which do not involve the changes in base sequence itself but regulate gene expression. DNA methylation is one of the most widely studied epigenetic changes and is frequently associated with carcinogenesis. Methylation affects chromatin packaging, resulting in transcriptional silencing of the associated genes. Cancer cells use this mechanism to silence tumor suppressor genes, including those associated with cell differentiation, cell-cycle regulation, apoptosis, and DNA repair response, and activate oncogenes that confer a survival and proliferative advantage to the cancer cells.

DNA methylation is an enzymatic reaction catalyzed by DNA methyltransferase (DNMT) that results in the formation of a covalent bond between the methyl group of S-adenosyl methionine (SAM) and the fifth position of cytosine of unmethylated CpG dinucleotide i.e. cytosines that are followed by guanines, in the gene promoter region. Thus, major sites of DNA methylation are CpG sites and the regions of the genome that are enriched in CpG sites are termed CpG islands. The human genome encodes five DNMTs, including DNMT1, DNMT2, DNMT3a, DNMT3b, and DNMT3L. Specifically, DNMT1 copies the methylation pattern during replication maintaining pre-existing methylation patterns in hemi-methylated DNA, DNMT3a and -3b catalyze de novo DNA methylation i.e. catalyze methylation on unmethylated DNA, DNMT2 acts as RNA methyltransferase and DNMT3L lacks any catalytic activity and acts as a regulator of other DNMTs. Methyl-binding proteins i.e. MBD1, MBD2, MBD4, and MeCP2 recognize and bind DNA at methylated sites. These MBDs undergo complex formation with other epigenetic enzymes, including histone methyltransferases and histone deacetylases that catalyze the histone modifications leading to chromatin compaction, which causes gene expression silencing.

Aberrant DNA methylation at the CpG islands occur genome-wide in AML. DNMT1, among all 5 DNMTs, is the most highly-expressed DNMT in replicating cells. Thus, DNMT1 inhibition is a target for inhibiting DNA methylation to restore tumor suppressor genes in rapidly dividing cancer cells. Moreover, in AML DNMIT1 is a potential oncoprotein. Azacytidine-resistant AML cells overexpress DNMT1 protein. Also, miRNAs targeting 3′UTR of DNMT1 are downregulated in AML resistant patients. Thus, DNMT1 is a promising therapeutic target in AML. In addition, methylation patterns of several genes, including p15INK4B (cyclin-dependent kinase inhibitor 2B), AWT1, BMI1 C1R, EZH2, HIC1, ID4, MGMT, RING1, sFR2, TERTpro/Ex1 have been associated with poor clinical outcome in AML.

The importance of aberrant hypermethylation in AML is demonstrated by the clinical success of two pyrimidine analogues that inhibit DNMT methylating activities (i.e., 5-aza-2′-deoxycytidine [decitabine]) and 5-azacitidine [azacitidine] to induce complete remissions in AML patients [1,2]. These nucleoside analogs are pro-drugs that undergo phosphorylation to the triphosphate nucleotide, and are incorporated into DNA during DNA replication to act as inhibitors of DNMT. These agents resemble cytosine and are thus able trap DNMT upon incorporation into DNA. DNMT1 undergoes a covalent bond formation with the carbon at 6^{th} position of the cytosine as well as that of cytosine analog, 5-aza-cytosine ring. Under physiological conditions, DNMT1 enzyme catalyzes the transfer of the methyl group from SAM to the carbon at position 5 of the cytosine ring. This reaction releases the enzyme from its covalent bond with cytosine. When the cytosine ring is substituted by 5′-aza-cytosine in the DNA, the methyl transfer fails to take place and the enzyme is trapped on the DNA. The replication fork progresses in the absence of DNMT1 activity leading to passive loss of DNA methylation in the newly synthesized nascent strand but not the template The trapped DNMTs are ultimately degraded by the proteasome leading to DNA hypomethylation.

The major difference between decitabine and 5-AC is that unlike decitabine that integrates only into DNA, 5-AC incorporates into both DNA and RNA. Intracellularly, approximately 85% of 5-AC is converted to its triphosphate form and 5-AC(CTP) is also incorporated into RNA to inhibit protein synthesis. About 10 to 20% of 5-AC is converted by ribonucleotide reductase to deoxyribose which is phosphorylated to 5-AC(dCTP) and incorporated into DNA. Regardless, the mechanisms of action, both the agents remain important drugs in the clinic for treatment of AML.

Importantly, hypermethylated tumor suppressor genes that are silenced in acute myeloid leukemia (AML) can be made accessible and reactivated by low, sub-cytotoxic dose of 5-AC. 5-AC depletes DNMT1, which facilitates differentiation and cell cycle exit across multiple AML subtypes irrespective of p53 status. The low dose of 5-AC is found to be efficacious and below the damaging threshold to stem cells and normal marrow as demonstrated by many studies and clinical trials.

However, only ˜50% of AML patients to achieve a meaningful clinical response with DNMT inhibitor treatment. Primary resistance, i.e. failure to demonstrate any initial response, or secondary response, i.e. acquired drug resistance following robust initial activity remains a major challenge with hypomethylating agents in the clinic. An important reason for the primary resistance to hypomethylating drugs is the enzyme cytidine deaminase (CDA), it rapidly metabolizes cytidine analogues, including 5-AC, through deamination into inactive uridine and dramatically reduces 5-AC half-life from hours to only few minutes. Some organs, such as the liver, intestines, and blood express high levels of CDA, thus adding more resistance and possibly providing sanctuary to cancer cells. CDA is also found to be upregulated within cancer cells. 5-AC could be eliminated even faster by CDA when it is given at low dosage. This may seriously counter the goal of sustaining low levels of 5-AC. An available solution to the problem is Tetrahydrouridine (THU), a competitive inhibitor of CDA. THU is a uridine analogue with wide safety index that has been used in combination with Hypo Methylation Agents (HMAs) for decades, to enhance DNMT1 depletion. several studies have shown that addition of TIHU increases the in-vivo stability of 5-AC. Combining THU with 5-AC can sustain low dose of 5-AC for a substantially longer time and promote better distribution. Another advantage is that inhibition of intestinal CDA could improve oral bioavailability for oral forms of HMAs and addresses possible sanctuary within all CDA rich tissue. This is significant as it could reduce post treatment minimal residual disease that usually leads to chemo-resistance within cancer cells and eventually relapse.

### SUMMARY

The disclosure provides a method of treating cancer or a blood disorder in a patient, the method comprising administering a therapeutically effective amount of a combination of compounds comprising (1) a hypomethylating agent (HMA) and (2) an XPO1 inhibitor to the patient.

Embodiments are described in which the XPO1 inhibitor is Valtrate or a derivative thereof or Caffeic acid phenethyl ester (CAPE) or a derivative thereof. The compounds may be administered as pharmaceutically acceptable salts of hydrates.

This disclosure provides methods in which the XPO1 inhibitor and the HMA are the only active agents or in which the XPO1 inhibitor (such as Valtrate or CAPE) and the HMA are administered together with one or more additional active agents.

Included in the embodiments are a method of treating cancer or a blood disorder in a patient, the method comprising administering a therapeutically effective amount of a combination comprising (1) a hypomethylating agent (HMA) and (2) a compound selected from Valtrate or a derivative thereof and Caffeic acid phenethyl ester (CAPE) or a derivative thereof, and the pharmaceutically acceptable salts and hydrates of any of the foregoing to the patient.

The embodiments also include a method of treating acute myeloid leukemia (AML) in a patient comprising administering a therapeutically effective amount of a combination of (1) a hypomethylating agent (HMA) and (2) an XPO1 inhibitor to the patient, wherein the patient is identified as having leukemia with either a MLL mutation or one or both of an NPM1 mutation and an FLT3 mutation.

### BRIEF DESCRIPTION OF THE DRAWINGS

FIG. 1A-FIG. 1J. These figures provide a summary of therapeutic approaches accomplished by overcoming the hypermethylated state of tumor suppressor genes (TSGs) (achieved by hypomethylating agents (HMAs)) combined with XPO1 inhibitors (shifting transcription/differentiation factors back to nucleus).: In normal hematopoiesis, balanced shifting of transcription and differentiation factors occurs between nucleus and cytoplasm during normal development (FIG. TA) and differentiation to a mature cell (FIG. 1B). In transformed cells closed chromatin/hypermethylation and shifting of transcription and differentiation factors from nucleus to cytoplasm (FIG. 1C) results in uncontrolled cell division (FIG. 1D). Treatment with HMAs (FIG. 1E) reduces hypermethylation resulting in only limited therapeutic success and a nucleas/cytoplasm imbalance of transcription and differentiation factors (FIG. 1F). Treatment with XPO-1 Inhibitor (FIG. 1G) can shift the transcription and differentiation factors back to the nucleus but does not address the closed hypermethylated chromatin (FIG. 1H). The combination of HMAs and XPO-1 Inhibitor (FIG. 1I) simultaneously tackles both closed chromatin/hypermethylation (and discytoblasmic shift promoting normal differentiation (FIG. 1J).

FIG. 2. In-silico screening analysis identifies valtrate and caffeic acid phenethyl ester (CAPE) as having high XPO-1 binding activity. (FIG. 2A) Valtrate and (FIG. 2B) CAPE show different types of interaction with XPO-1 protein at multiple amino acid residues. Three-dimensional (3D) structure of XPO-1 protein is shown.

FIG. 3. AML cell line differentiation in vitro is activated by nuclear export inhibitors and HMA(5-AC). Combination of Valtrate or CAPE with 5-AC induces synergistic differentiation. Various maturation morphologies of terminal fate are observed with the Valtrate+5-AC combination compared with CAPE or selinexor in combination with 5-AC.

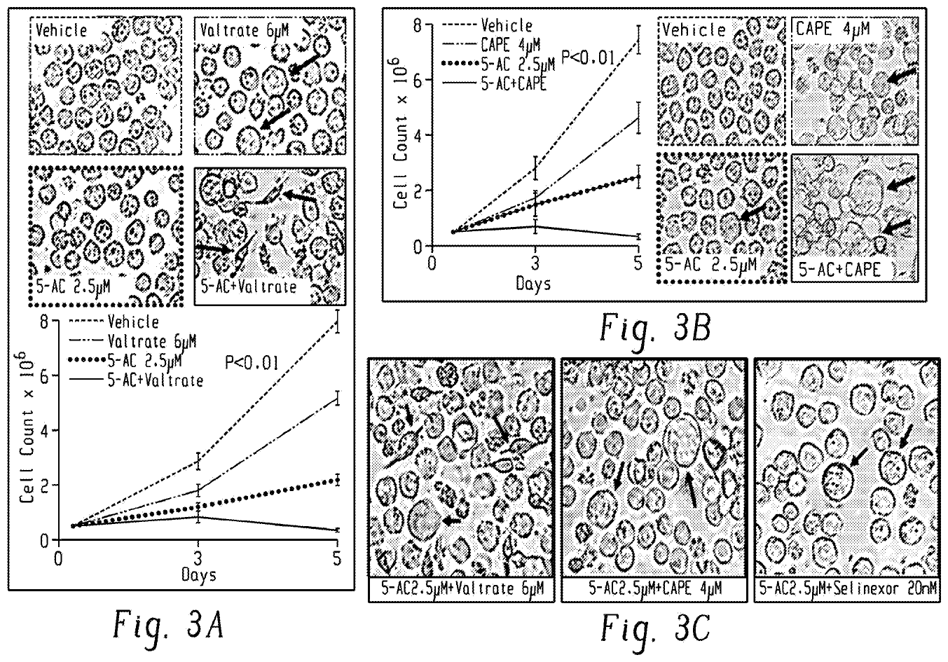

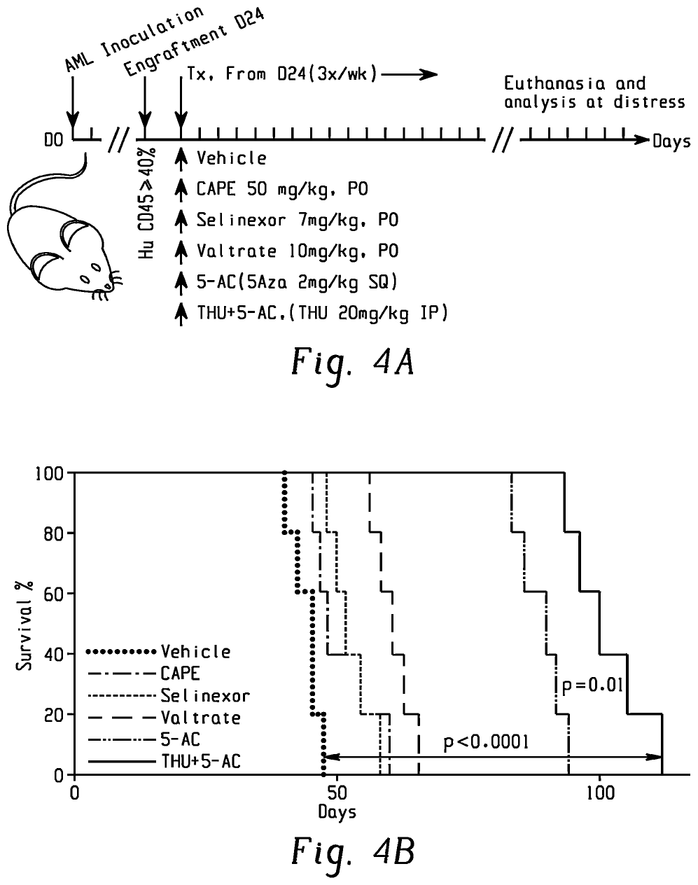

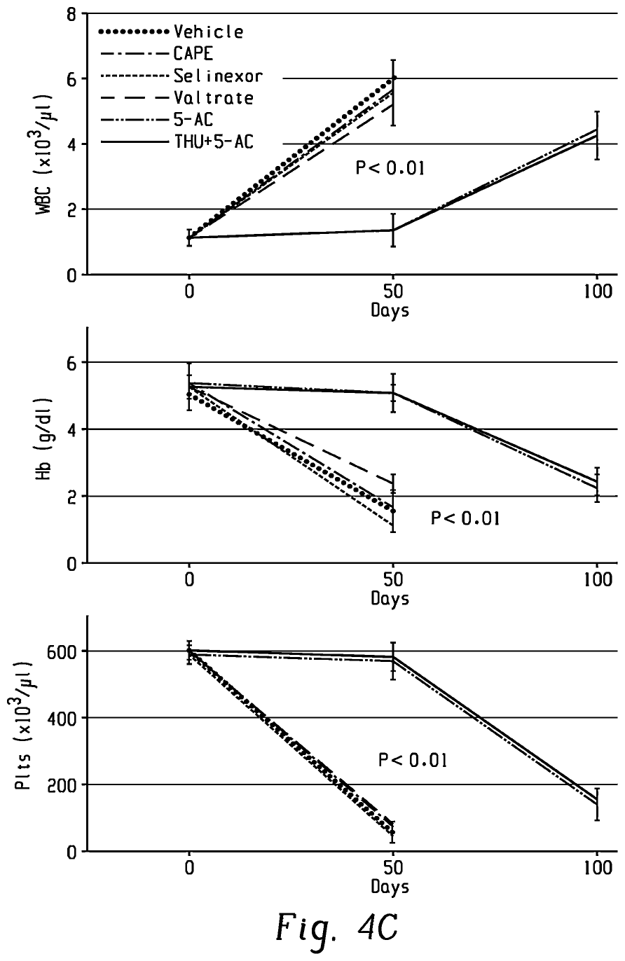

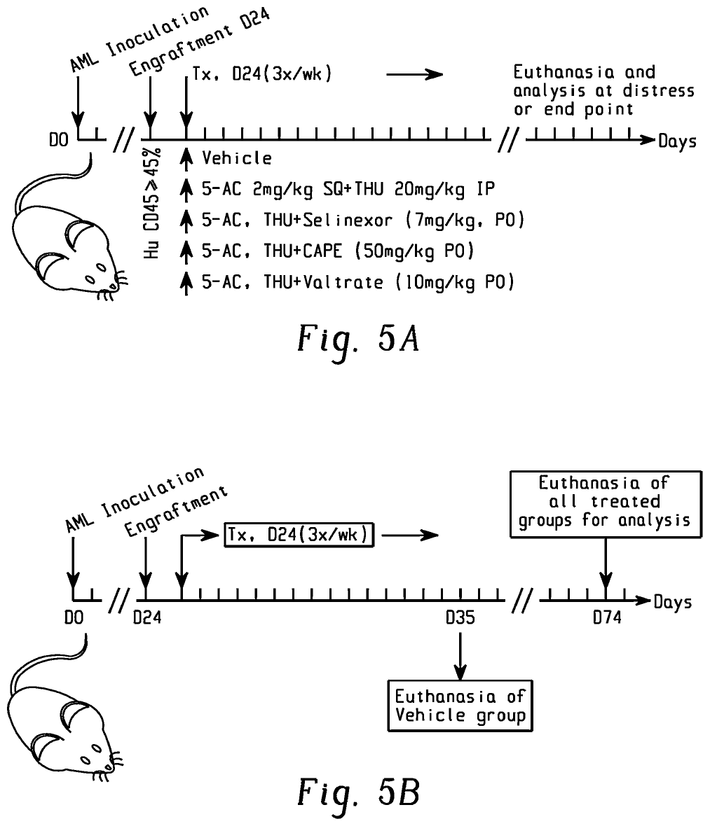

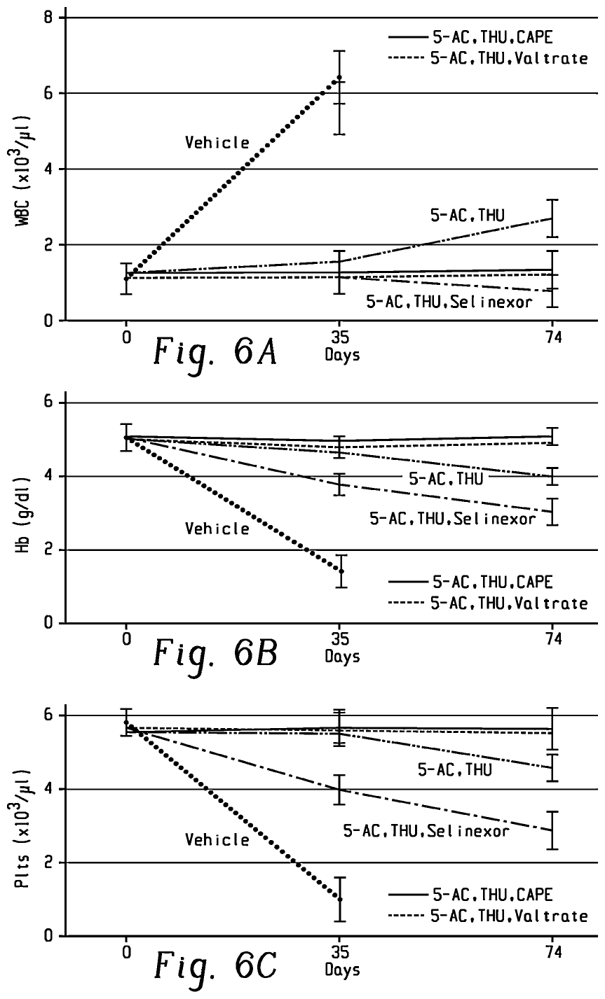

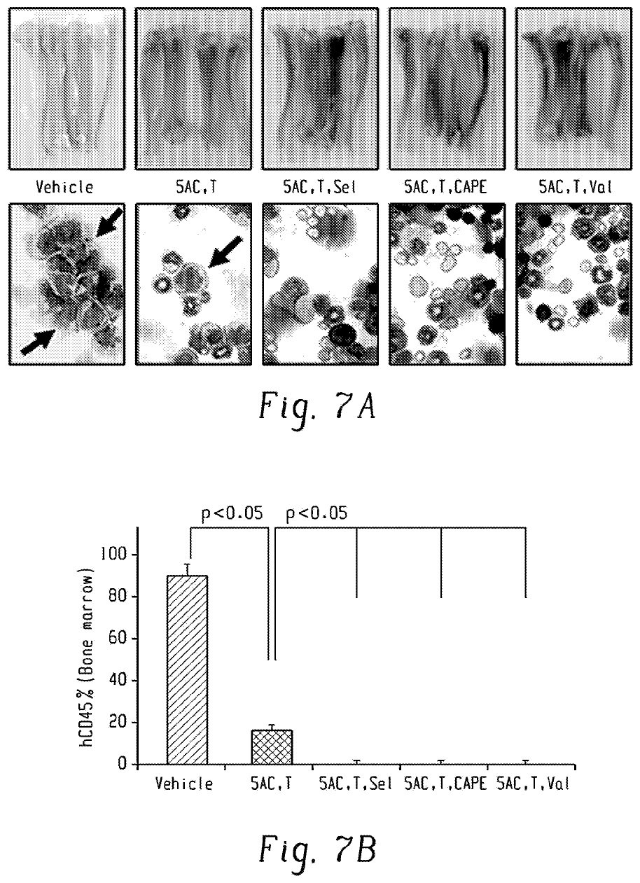

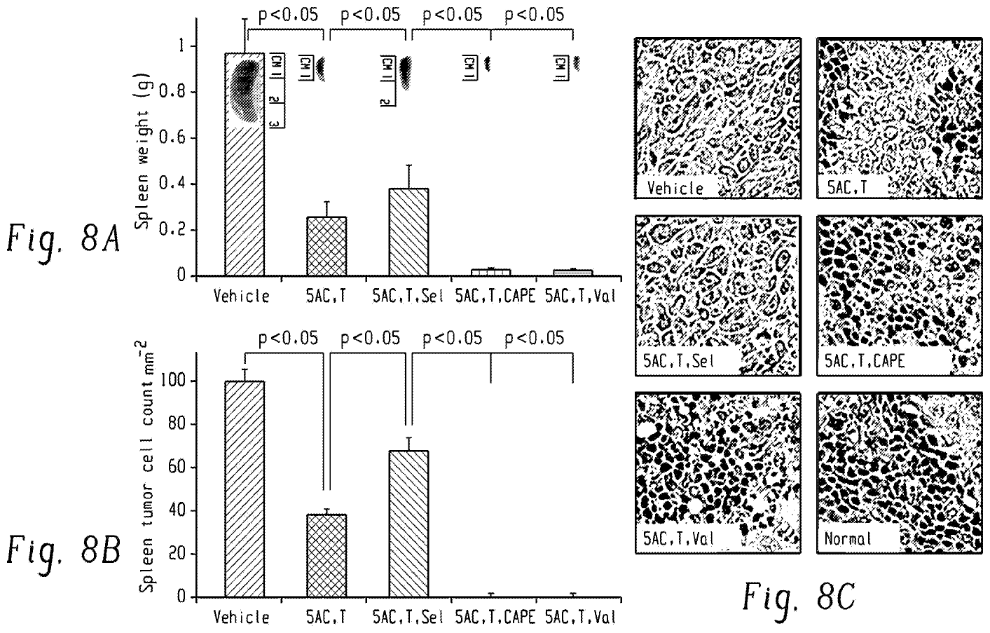

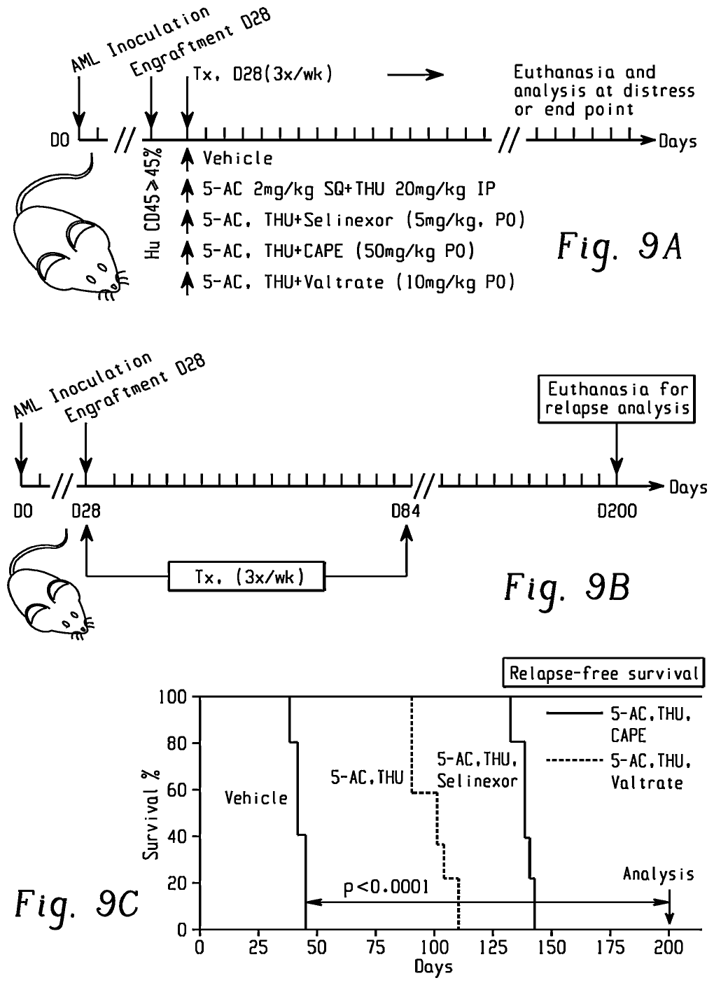

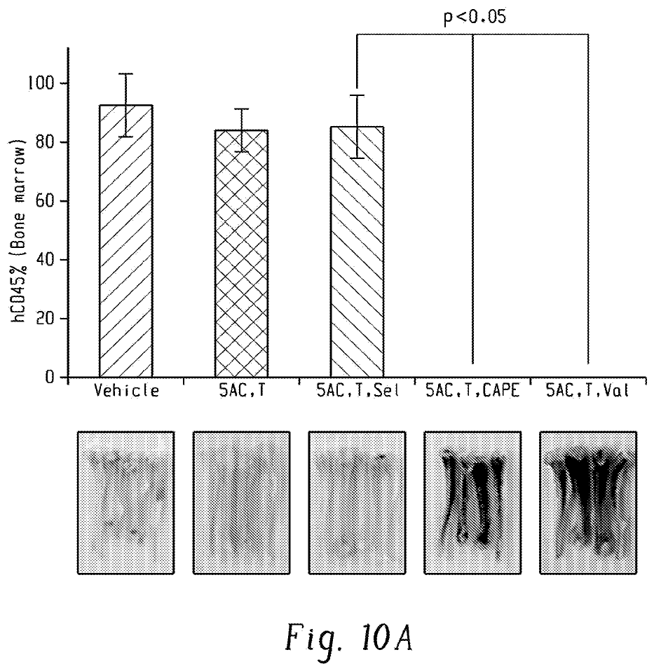

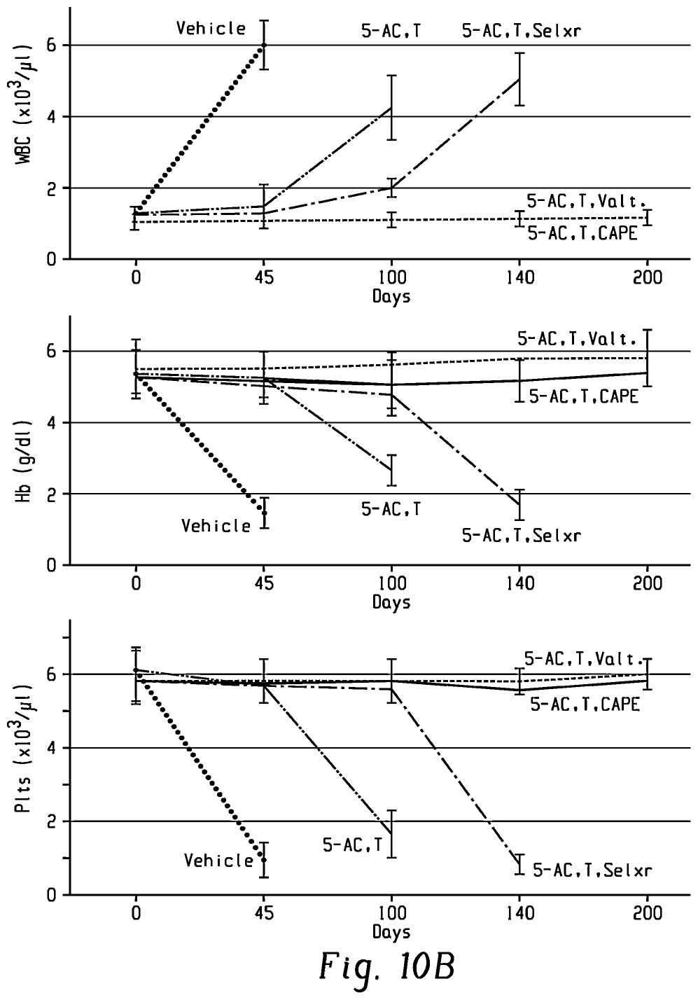

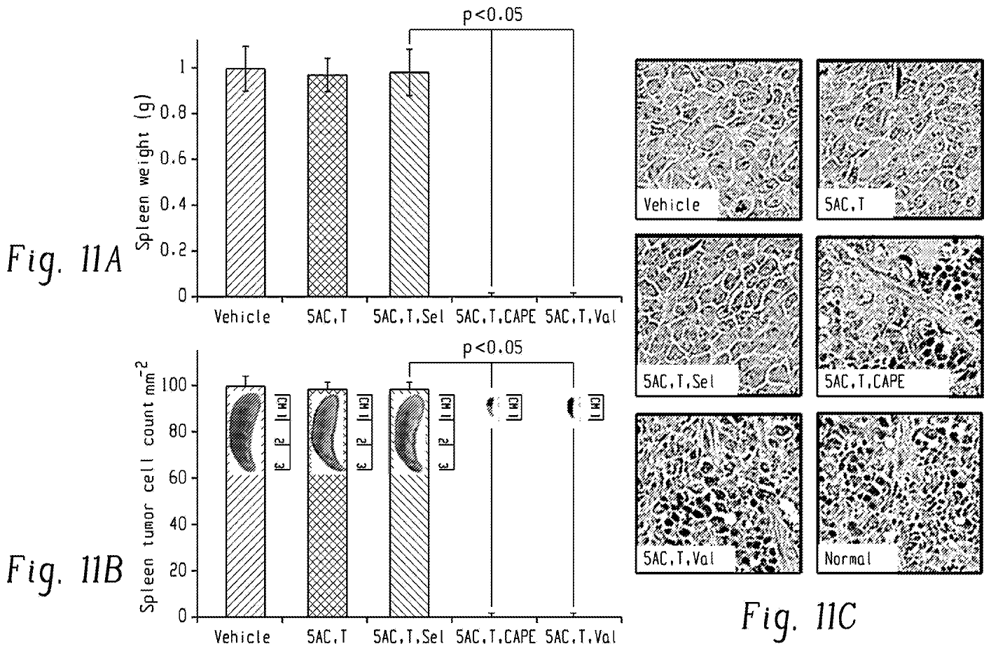

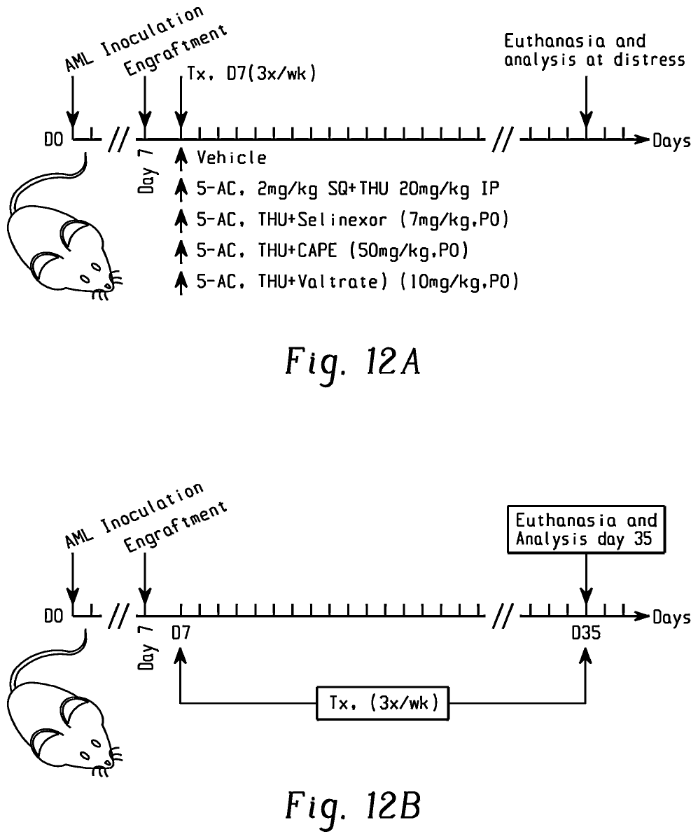

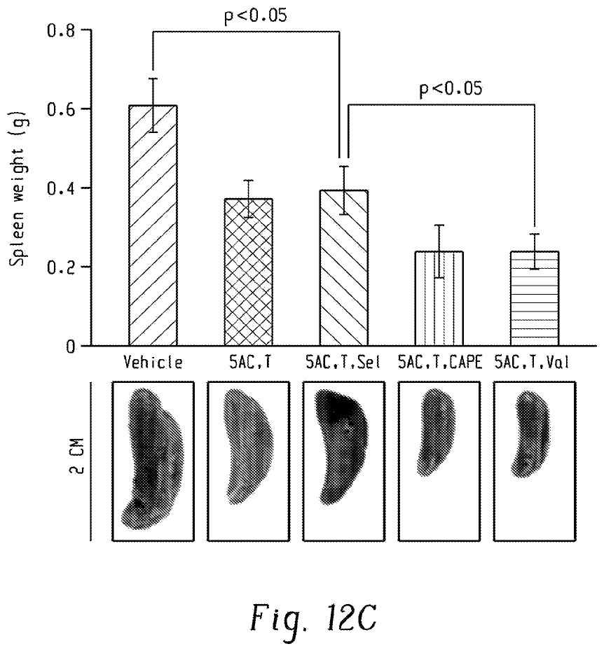

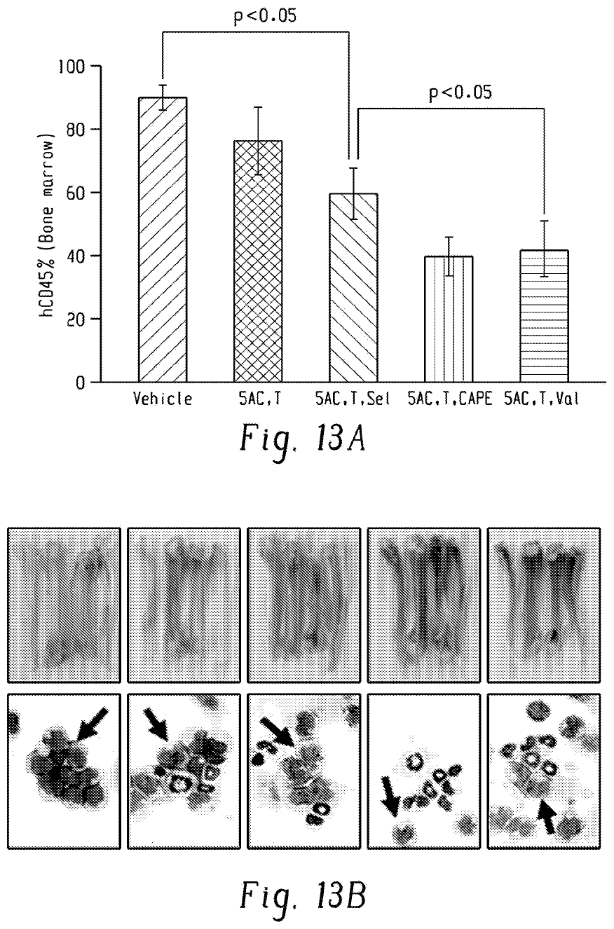

AML cell counts and images for morphologic differentiation of HL-60 cell line, 5-AC was used to induce hypomethylation by depleting DNMT1. Cell counts obtained using automated counter, Mean±SD (3 experiments) cell morphology obtained from the same cell culture. P<0.01 significant, t test (2 sided). The small molecule agents were used in dosages: Valtrate (6 μM), CAPE (4 μM), 5-AC (2.5 μM), selinexor (20 nM) and in the combinations relative to vehicle. cell counts (day 0-5) and morphology (day 5). (FIG. 3A) Valtrate, 5-AC, and Valtrate+5-AC cell count and morphologic changes relative to vehicle and individual agents. (FIG. 3B) CAPE, 5-AC, and CAPE+5-AC cell counts day 0-5 and morphologic changes on day 5 relative to vehicle and individual agents. (FIG. 3C) Combinational groups of 5-AC+valtrate, 5-AC+CAPE, and 5-AC+selinexor morphologic development (day 5) are compared.

FIG. 4. The in vivo effect on survival of single agent treatment of mice with Valtrate, CAPE, Selinexor, 5-AC, and 5-AC in Combination with THU, in a Patient-Derived Mouse Xenograft Model with NPM1/FLT3 Mutations.

FIG. 4A. Experiment plan diagram. Primary acute myelogenous leukemia (AML) patient cells identified as having Nucleophosmin 1 and fins-like tyrosine kinase-3 mutations (NPM1/FLT3) were xenotranplanted into immunodeficient Nod-SCID-IL-2Rgamma-null mice (NSG, Jackson Laboratory) by injecting 1×10⁶ cells IV (n=5 per group) at day 0 (D0). At day 24≥40% bone engraftment was confirmed in 3 randomly selected mice. At day 24 mice were divided in to six treatment groups (n=5 per group), 1) Vehicle control buffer, 2) Valtrate 10 mg/kg by oral gavage, 3) CAPE 50 mg/kg by oral gavage, 4) selinexor 7 mg/kg by oral gavage, 5-AC2 mg/kg subcutaneously, and 6) 5-AC(2 mg/kg) subcutaneously/THU (20 mg/kg) intraperitoneally. THU inhibits cytidine deaminase(CDA) to prolong 5-AC half-life. Treatment was initiated at day 24 and then repeated three times a week. Mice were humanly euthanized after becoming moribund or losing 15% of their initial weight in accordance with institutional guidelines. (FIG. 4B) Survival presented as Kaplan-Meier survival analysis graph from inoculation time to distress. P values and log-rank test are calculated and plotted. (FIG. 4C) Peripheral blood obtained by tail vein phlebotomy and the serial blood count analysis by HemaVet blood lab (Drew Scientific). Mean±SD. P<0.01 significant. The increase in WBCs was due to pooling of myeloblasts from bone marrow and spleen into blood stream.

FIG. 5. Combination of 5-AC/THU with the nuclear transport inhibitors Valtrate, CAPE, and Selinexor in fixed time point in vivo PDX Model—NPM1/FLT3 Mutation

FIG. 5A. Model and treatment plan graph: Primary acute myelogenous leukemia (AML) patient cells with Nucleophosmin 1 and fins-like tyrosine kinase-3 mutations (NPM1/FLT3) were xenotranplanted into immunodeficient Nod-SCID-IL-2Rgamma-null mice (NSG, Jackson Laboratory) by injecting 2×10⁶ cells IV (n=5 per group) at day 0 (D0). Engraftment D24≥40% bone engraftment was confirmed in 3 randomly selected mice. At D24 mice were divided in to five treatment groups (n=5 per group), 1) Vehicle, 2) 5-AC(2 mg/kg)/THU(20 mg/kg), 3) 5-AC(2 mg/kg)/THU(20 mg/kg)/selinexor (7 mg/kg), 4) 5-AC(2 mg/kg)/THU(20 mg/kg)/CAPE (50 mg/kg) and 5) 5-AC(2 mg/kg)/THU(20 mg/kg)/Valtrate(10 mg/kg). Treatment was initiated D24 and repeated three times a week.

FIG. 5B. Display of fixed time point euthanasia of groups relative to inoculation and treatment plan, vehicle group euthanized on average day 35 after becoming moribund in accordance with institutional guidelines. In order to allow longer treatment time and improved analytic measurement discrimination between treatment groups euthanasia of treated groups was set at fixed time point on day 74.

FIG. 6. Blood count days 0-74 in in vivo PDX Model—NPM1/FLT3 Mutation.

Peripheral blood analysis for white blood cells count WBCs (FIG. 6A), hemoglobin Hb (FIG. 6B), and platelets count (FIG. 6C). Peripheral blood obtained by tail vein phlebotomy and serial blood count on day 0, day 35, and day 74 were analyzed by HemaVet blood lab. Mean±SD. P<0.01 significant.

FIG. 7. Bone marrow analysis of the 5-AC/THU combination with Valtrate, CAPE and Selinexor in vivo PDX Model—NPM1/FLT3 Mutation.

Femoral and tibial bones were removed from each mouse. White bones indicate leukemia replacement has occurred, red bones, which appear darker in the black and white photographs, indicate functional hematopoiesis. (FIG. 7A, top) Bone marrow myeloid content was evaluated by Giemsa staining bone marrow cells for evaluation (FIG. 7A, bottom). Flow cytometry was used to determine human (hCD45) tumor load percentage in mouse bone marrow Median±IQR. p value Mann-Whitney test two-sided (FIG. 7B).

FIG. 8. Spleen analysis of the 5-AC/THU combination with Valtrate, CAPE and Selinexor in vivo PDX Model—NPM1/FLT3 Mutation Spleens were removed, photographed, and the tumor burden of infiltrating AML cells was represented by spleen weight (FIG. 8A). An image analysis approach to further evaluate tumor burden of the spleens, was by histologic identification of the relatively large infiltrating AML cells by H&E (Hematoxylin and eosin) staining of spleen sections to determine the density and objectively define distribution patterns. Image analysis of cell counts were plotted and quantified (FIG. 8B). H&E staining of spleen sections for vehicle and treatment groups are shown, a normal NSG mouse spleen section was added for reference (FIG. 8C).

FIG. 9. in vivo survival test of treatment combinations of 5-AC/THU with Valtrate, CAPE or Selinexor in PDX Model of NPM1/FLT3 Mutation

FIG. 9A Treatment groups illustration: Primary acute myelogenous leukemia (AML) patient cells with Nucleophosmin 1 and fins-like tyrosine kinase-3 mutations (NPM1/FLT3) were xenotranplanted into immunodeficient Nod-SCID-IL-2Rgamma-null mice (NSG, Jackson Laboratory) by injecting 1×10⁶ cells IV (n=5 per group) at day 0 (D0). Engraftment D28≥45% bone engraftment was confirmed in 3 randomly selected mice. At D28 mice were divided in to five treatment groups (n=5 per group), 1) Vehicle, 2) 5-AC(2 mg/kg)/THU(20 mg/kg), 3) 5-AC(2 mg/kg)/THU(20 mg/kg)/selinexor (5 mg/kg), 4) 5-AC(2 mg/kg)/THU(20 mg/kg)/CAPE (50 mg/kg) and 5) 5-AC(2 mg/kg)/THU(20 mg/kg)/Valtrate(10 mg/kg).

FIG. 9B. Treatment frame and terminal analysis design. Treatment was initiated on day 28, repeated three times a week and terminated on day 84. Vehicle group euthanized on average day 46 at distress signs in accordance with institutional guidelines. In order to observe survival of groups with or without relapse treatment was concluded on day 84. Euthanasia of groups was set at distress or at a fixed time point, 116 days post treatment cessation.

FIG. 9C. Groups survival is presented as, Kaplan-Meier survival analysis graph, from inoculation time to distress, and relapse free groups are indicated with terminal analysis point. P values and log-rank test are calculated and plotted.

FIG. 10. Bone marrow and Peripheral blood analysis of survival experiment of treatment combinations of 5-AC/THU with Valtrate, CAPE, or Selinexor in the PDX Model of NPM1/FLT3 Mutation

FIG. 10A. Femoral and tibial bones were removed and photographed from each mouse. White bones indicate leukemia replacement has occurred, and darker bones indicate functional hematopoiesis. (FIG. 10 A bottom) Flow cytometry was used to determine human (hCD45) tumor load percentage in mouse bone marrow Median±IQR. p value Mann-Whitney test two-sided (FIG. 10 A top).

FIG. 10B. Peripheral blood analysis of WBCs, hemoglobin Hb, and platelets count. peripheral blood obtained by tail vein phlebotomy and serial blood counted on day 0, 45, 100, 140 and 200. And analyzed by HemaVet blood lab. Mean±SD. P<0.01 significant.

FIG. 11. Extramedullary tumor burden measured in spleens of groups in survival experiment treatment combinations 5-AC/THU with Valtrate, CAPE, or Selinexor in PDX Model of NPM1/FLT3 Mutation

FIG. 11 A. Spleens were removed, photographed, and the tumor burden of infiltrating AML cells was represented by spleen weight.

FIG. 11B. An image analysis to evaluate tumor burden of the spleens, was by histologic identification of relatively large infiltrating AML cells, replacing normal splenic structure by H&E staining of spleen sections. Image analysis of cell counts were plotted and quantified.

FIG. 11C. H&E staining of spleen sections for vehicle and treatment groups are shown, a normal NSG mouse spleen section was added for reference.

FIG. 12. To further validate the efficacy of combinational treatments additional aggressive PDX model of MLL mutations is planned for fixed time point analysis

FIG. 12A. treatment plan, using the same experimental design, mouse xenograft experiments were performed with mixed-lineage leukemia (MLL)-mutated primary AML cells. 1×10⁶ cells were injected IV (n=5 per group) at day 0 (D0). Engraftment was confirmed in 3 randomly selected mice. At D7 mice were divided in to five treatment groups (n=5 per group), 1) Vehicle, 2) 5-AC(2 mg/kg)/THU(20 mg/kg), 3) 5-AC(2 mg/kg)/THU(20 mg/kg)/selinexor (5 mg/kg), 4) 5-AC(2 mg/kg)/THU(20 mg/kg)/CAPE (50 mg/kg) and 5) 5-AC(2 mg/kg)/THU(20 mg/kg)/Valtrate(10 mg/kg).

FIG. 12B. scheme of fixed time point euthanasia relative to inoculation and treatment plan. Being a very aggressive model, Bone marrow engraftment was confirmed at D7 rather than D24. At D35 mice were humanly euthanized after vehicle group become moribund or losing 15% of their initial weight in accordance with institutional guidelines.

FIG. 12C. Spleens were removed, fixed, photographed, and the tumor burden of infiltrating AML cells was represented by spleen weight.

FIG. 13. Bone marrow analysis of PDX model of MLL mutations

FIG. 13A. Flow cytometry to define human (hCD45) tumor load percentage in mouse bone marrow Median±IQR. p value Mann-Whitney test two-sided.

FIG. 13B. photographs of femoral and tibial bones were removed from each mouse. White bones indicate leukemia replacement has occurred; darker marrow bones indicate functional hematopoiesis. (FIG. 13B. top) Bone marrow myeloid content was evaluated by Giemsa staining bone marrow cells for evaluation (FIG. 13B. bottom)

### DETAILED DESCRIPTION

Prior to describing the invention in detail, it is helpful to consider the following definitions.

### Terminology

Compounds are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced items. The term “or” means “and/or”. The terms “comprising,” “having,” “including,” and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim.

All compounds are understood to include all possible isotopes of atoms occurring in the compounds. Isotopes include those atoms having the same atomic number but different mass numbers. The disclosure includes methods in which one or both of the XPO1 inhibitor or HMA are isotopically enriched. For example any of Valtrate, CAPE, or 5-AC can be isotopically enriched with a non-radioactive isotope at one or more positions. By way of general example, and without limitation, isotopes of hydrogen include tritium and deuterium and isotopes of carbon include ¹¹C, ¹³C, and ¹⁴C.

The opened ended term “comprising” includes the intermediate and closed terms “consisting essentially of” and “consisting of” Wherever an open ended embodiment that may contain additional elements is contemplated (comprising language), more narrow embodiments that contain only the listed items (consisting of language) are also contemplated.

“Pharmaceutical compositions” means compositions comprising at least one active agent, such as a Valtrate, CAPE, or an HMA, and at least one other substance, such as a carrier. Pharmaceutical compositions meet the U.S. FDA's GMP (good manufacturing practice) standards for human or non-human drugs.

“Carrier” means a diluent, excipient, or vehicle with which an active compound is administered. A “pharmaceutically acceptable carrier” means a substance, e.g., excipient, diluent, or vehicle, that is useful in preparing a pharmaceutical composition that is generally safe, non-toxic and neither biologically nor otherwise undesirable, and includes a carrier that is acceptable for veterinary use as well as human pharmaceutical use. A “pharmaceutically acceptable carrier” includes both one and more than one such carrier.

A “patient” means a human or non-human animal in need of medical treatment. Medical treatment can include treatment of an existing condition, such as a disease or disorder or diagnostic treatment. In some embodiments the patient is a human patient.

“Providing” means giving, administering, selling, distributing, transferring (for profit or not), manufacturing, compounding, or dispensing.

“Administering” means giving, providing, applying, or dispensing by any suitable route. Administration of the combination includes administration of the combination in a single formulation or unit dosage form, administration of the individual agents of the combination concurrently but separately, or administration of the individual agents of the combination sequentially by any suitable route. The dosage of the individual agents of the combination may require more frequent administration of one of the agent(s) as compared to the other agent(s) in the combination. Therefore, to permit appropriate dosing, packaged pharmaceutical products may contain one or more dosage forms that contain the combination of agents, and one or more dosage forms that contain one of the combination of agents, but not the other agent(s) of the combination.

“Treatment” or “treating” means providing an active compound to a patient in an amount sufficient to measurably reduce any cancer symptom, slow cancer progression or cause cancer regression. In certain embodiments treatment of the cancer may be commenced before the patient presents symptoms of the disease.

A “therapeutically effective amount” of a pharmaceutical composition means an amount effective, when administered to a patient, to provide a therapeutic benefit such as an amelioration of symptoms, decrease cancer progression, or cause cancer regression.

A significant change is any detectable change that is statistically significant in a standard parametric test of statistical significance such as Student's T-test, where p<0.05.

“Derivative” of Valtrate or Caffeic acid phenethyl ester (CAPE) means any chemical modification to the structure of Valtrate or Caffeic acid phenethyl ester such as acid, ester, amide, or anhydride.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, or in separate containers (e.g., capsules) for each active ingredient. In addition, such administration also encompasses use of each type of therapeutic agent in a sequential manner, either at approximately the same time or at different times. In either case, the treatment regimen will provide beneficial effects of the drug combination in treating the conditions or disorders described herein.

“Pharmaceutically acceptable salts” include derivatives of the disclosed compounds in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media such as ether, ethyl acetate, ethanol, isopropanol, or acetonitrile are used, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in G. Steffen Paulekuhn, et al., Journal of Medicinal Chemistry 2007, 50, 6665 and Handbook of Pharmaceutically Acceptable Salts: Properties, Selection and Use, P. Heinrich Stahl and Camille G. Wermuth, Editors, Wiley-VCH, 2002.

### Pharmaceutical Description

The XPO1 inhibitor can be Valtrate (CAS Reg. No. 18296-44-1) having the chemical formula

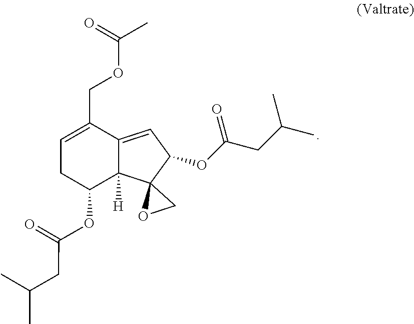

The XPO1 inhibitor can also be Caffeic Acid Phenethyl Ester (CAPE) (CAS Reg. No. 104594-70-9).

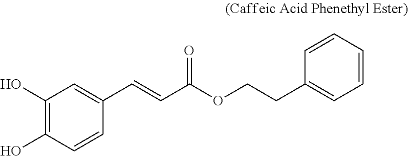 [SMILES: O=C(/C=C/c1ccc(O)c(O)c1)OCCc1ccccc1]

Oncogenic transformations involve complex events of genetic mutations and epigenetic dysregulations. Many cancers including hematological malignancies resist therapy irrespective of drug mechanism of action. This is in part due to disordered nuclear-cytoplasmic transport that shifts tumor suppresser proteins out of the nucleus, reduces treatment efficacy and leads to poor treatment outcomes. XPO1 is a key mediator of nuclear export that is highly expressed in many cancers.

Another predictor of poor outcome is the hypermethylation found in the promoters of tumor suppressor genes (TSGs) that has been reported in many cancers including AML. It is highly probable that monotherapy approach with either XPO-1 inhibitors or hypomethylation agents (HMAs), tackles either the disordered nuclear-cytoplasmic transport or the hypermethylated genes, thereby having limited success.

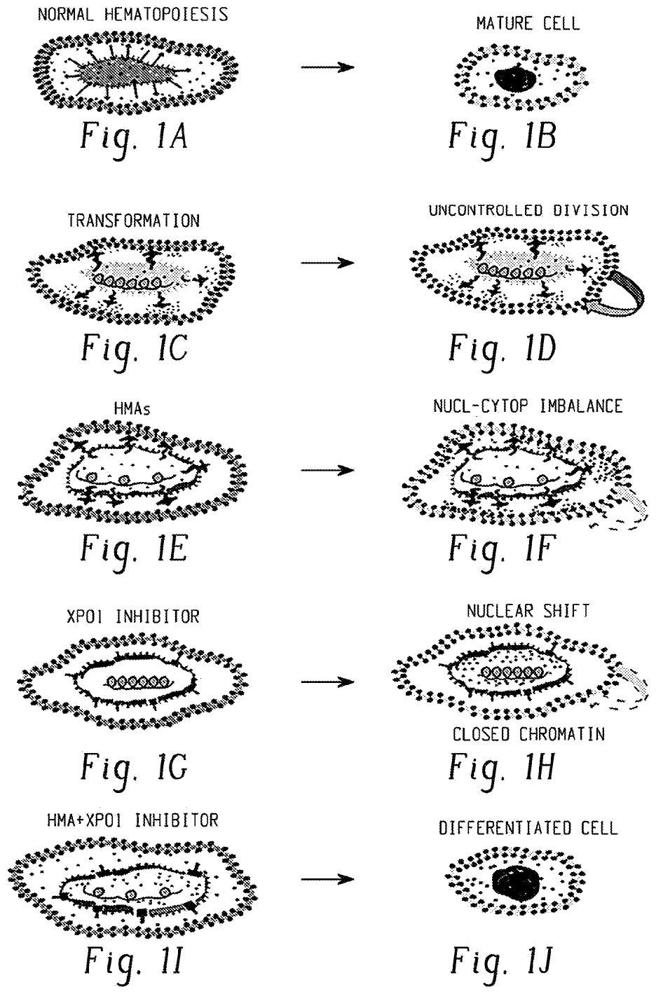

For decades, these critical aspects of malignancy were therapeutically addressed individually. We disclose a new combinational strategy to counter both the disordered nuclear-cytoplasmic transport and the hypermethylated state of TSGs simultaneously (FIG. 1).

An aspect of this disclosure is the provision of a safe and effective molecule that inhibits the activity of XPO-1. XPO-1 inhibition with the molecule counters disordered nuclear-cytoplasmic transport. In normal cells, a delicate balance is sustained by continuously shuttling proteins and RNA in and out of the nucleus. This transport system uses a set of specialized proteins that include importins (for import), exportins (for export) and transportins (for both). XPO1 (Exportin-1/Chromosome Region Maintenance 1/CRM1) is a key mediator of nuclear protein export in many cell types that mainly controls their position and function. XPO1 is upregulated and utilized by many cancers including leukemia to delocalize tumor suppressors, cell cycle regulators, and transcription factors from nucleus to cytoplasm. It is also associated with poor prognosis. In that context, XPO1 represented an appropriate target in cancer treatment. Structure-activity relationship studies of recognition of NES by XPO1 have led to identification and development of several selective inhibitors of nuclear export (SINE)s through in silico small molecule docking screens. SINEs are reversible inhibitors that undergo covalent bond formation with the critical Cys528 residue within the NES-binding groove of XPO1. This binding hinders the recognition of XPO1 cargos through NES and, therefore, the export of the nuclear cargos into the cytoplasm is inhibited. This ends up concentrating the XPO1 cargos, for example p53, MDM2, p27, mTOR, p73, PAR-4, topoisomerase IIα, IκB, and NPM1, in the nucleus which regulate gene expression to promote cell-cycle arrest, apoptosis, DNA damage response, and anti-tumor immunity with an overall anti-cancer effect [8]. Substantial pre-clinical data exists to support the efficacy of SINEs across a wide spectrum of malignancies.

selinexor, a first-in-class selective inhibitor of XPO1, has already been approved by U.S. Food and Drug Administration (FDA) for multiple myeloma and diffused cell large cell lymphoma. However, the clinical applicability of selinexor has been limited by the treatment-emergent undesirable side effects. Hematological toxicities, including thrombocytopenia, anemia, and neutropenia are commonly associated with selinexor treatment, and often require close monitoring. The underlying cause of these side-effects include poor bone marrow reserve, particularly in previously treated patients, impaired thrombopoietin signaling and differentiation of stem cells into megakaryocytes. The most common non-hematological toxicities include fatigue, hyponatremia, nausea, vomiting, and diarrhea which are a class effect of the medication.

Further, even though XPO1 inhibitors restore mislocalized growth suppressors and TFs back to the nucleus. It is highly probable that these factors cannot reach the promotor sites of hypermethylated inaccessible TSGs. XPO1 inhibitors as monotherapy do not tackle this aspect of hypermethylated genes, and may not reach their full therapeutic potential for this reason.

The disclosure provides a means to overcome the hypermethylated state of TSGs. In this aspect the DNA methylation enzyme DNMT1 is targeted with FDA approved hypomethylating agents (HMAs) 5-Azacytidine (5-AC), and 5-aza-2′-deoxycitidine (DAC). Both of these agents have an established prognostic importance especially in patients with myelodysplastic syndromes (MDS) and AML. HMAs are well tolerated and usually given as six cycles for better response. Nonetheless, most patients show hematologic improvement but only a minority achieve complete response. In addition, these prolonged treatments obviously contribute to development of chemo-resistance. Despite the fact that HMAs render TSGs promoters more accessible for transcription factors (TFs) and transcriptional machinery of coactivators to initiate transcription. Unfortunately, because of cytoplasmic displacement, TFs may not be available in high enough concentration to trigger the transcription process in the nucleus. More importantly, HMAs as monotherapy do not engage the critical aberrant nuclear-cytoplasmic imbalance. This is particularly important since tumor growth suppressors that regulates cell division are delocalized into the cytoplasm to become functionally inactive. Examples for these key transcription factors are P53, p21, P27, FOXO, RUNX3, APC, NPM1, and Fbw7γ.

Such onocogenic alterations cooperate in a striking manner to promote growth and evade treatment response. Tumor suppressor gene promotors are silenced and made inaccessible by hypermethylation. And, the mislocalized nuclear cell cycle inhibitors, TFs, and differentiation factors are targeted to the cytoplasm to be degraded by proteasome. In cancer cells these events are lineage specific, impacted by the mutational landscape. Taken together this could explain why monotherapies targeting one aspect and not the other usually resulted in longer drug exposure time which increases both chemo-resistance and toxicities while limiting therapeutic potential.

Therefore, our therapeutic approach presents an overlapping of phases, during which shifting transcription factors back to nucleus (achieved by XPO1 inhibitors) is met with accessible promotor sites of tumor suppressor and differentiation genes (achieved by HMAs) (FIG. 1).

When aiming to correct multiple events in parallel, combinational therapy would be more effective, minimize drugs exposure time, reduce toxicities and even cost effective. Integrating an effective drug combination, however, is challenging even when relevant targets are established. Factors such as therapeutic index and untoward or unidentified off-target effects of different compounds could undermine the combination purpose. Therefore our criteria was to identify natural agents that work in synergistic manner, be given orally at low non-cytotoxic dose without compromising efficacy, promote differentiation, and terminate proliferation independent of apoptotic pathway. To achieve this objective in AML we are combining HMA (5-AC) with XPO1 inhibitors.

An extended list of XPO1 inhibitors was identified by investigators and later refined for better efficacy. Newer generations has also been developed and evaluated for cancer treatment [11, 13] but yet showed limited clinical success and substantial site effects. XPO1 exports high number of proteins besides tumor suppressors, furthermore it is involved indirectly in other functions that leads to upregulation of oncogenes such as HIF-1, c-Myc and vascular endothelial growth factor. Inhibiting XPO1 can influence several events and pathways in tumor cell. Taking into consideration these possibilities of variables, we developed a selection criteria for XPO1 inhibitors that took into account: in silico docking analysis, in vitro evidence of nuclear balance shift of tumor suppressors, oral administration with well tolerated profile and most importantly in vivo synergistic action with HMAs at dosages that does not cause myelosuppression or peripheral cytopenias.

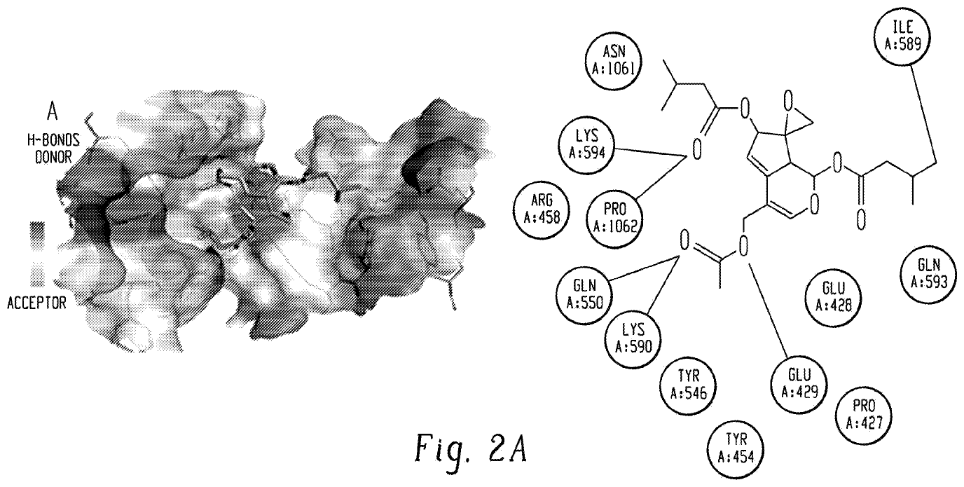

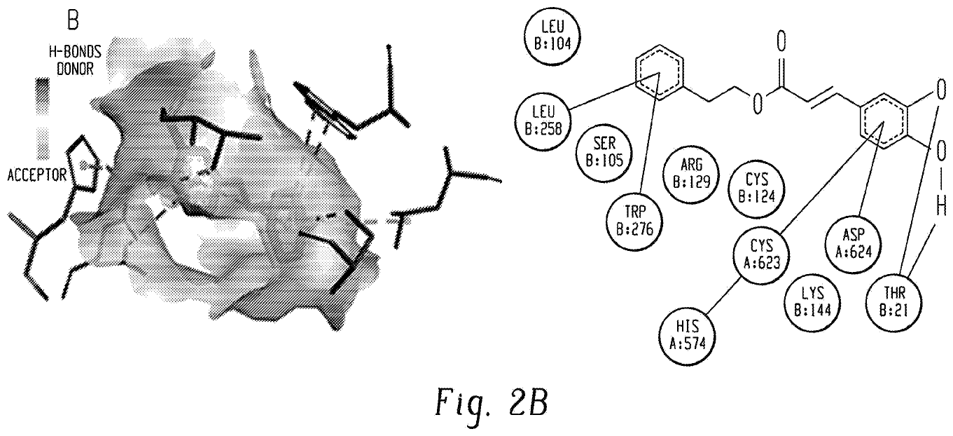

Our research yielded two small-molecule inhibitors that are natural compounds from plant sources: Valtrate and Caffeic acid phenethyl ester (CAPE) (FIG. 2), both of which work synergistically with 5-AC and meet our criteria.

Our in silico docking analysis shows that Valtrate, which is a traditional Chinese medicine isolated from valeriana fauriei and used to treat mental disorders, has a strong binding activity to XPO-1 (FIG. 2A). valtrate has been reported to have antiviral effect by inhibiting influenza virus replication. Our in silico docking analysis also showed that Caffeic Acid Phenethyl Ester (CAPE) has a strong binding activity to XPO-1 (FIG. 2B). CAPE is found in several types of plants as well as honeybee hives. Studies investigating CAPE show anticarcinogenic, immune system modulating and reduced inflammatory response. We tested Valtrate and CAPE orally to establish dosage safety, efficacy and most importantly synergy with 5-AC (2 mg/kg)/THU(20 mg/kg) in an in-vivo PDX mouse models of AML (NPM1-FLT3 and MLL) we established a nontoxic pharmacologically effective combinational doses (FIGS. 3,4,5). Our result indicate that 5-AC/THU in combination with oral Valtrate (10-50 mg/kg) or oral CAPE (10-50 mg/kg) are noncytotoxic, well tolerated and eliminate AML cells in bone marrow and spleen in PDX model of AML (NPM1-FLT3 and MLL) (FIGS. 5,6,7,8,9). Our data show that oral Valtrate (10 mg/kg·3×/wk) or CAPE (50 mg/kg·3×/wk) provide an effective synergetic action in combination with 5-AC (FIGS. 5,6,7,8,9).

Pharmaceutical Compositions

Valtrate, or Caffeic acid phenethyl ester (CAPE), and the pharmaceutically acceptable salts and hydrates of any of the foregoing can be administered as neat chemicals but are preferably administered as a pharmaceutical composition. Accordingly, the disclosure provides pharmaceutical compositions comprising a first active compound selected from any of Valtrate, Caffeic acid phenethyl ester, and the pharmaceutically acceptable salts and hydrates together with at least one pharmaceutically acceptable carrier. The pharmaceutical composition may contain a compound or salt of XPO1 inhibitor and HMA are the only active agents, or may contain one or more additional active agents.

The first active compound may be administered orally, topically, parenterally, by inhalation or spray, sublingually, transdermally, intravenously, intrathecally, via buccal administration, or rectally, or by other means, in dosage unit formulations containing conventional pharmaceutically acceptable carriers. In certain embodiments the first active compound is administered orally. In certain embodiments the first active compound is administered subcutaneously or intravenously. The pharmaceutical composition may be formulated as any pharmaceutically useful form, e.g., as an aerosol, a cream, a gel, a pill, a capsule, a tablet, a syrup, a transdermal patch, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidents, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the compound of the present invention.

The pharmaceutical compositions can be formulated for oral administration. These compositions contain between 0.1 and 99 weight % (wt. %) of a compound of and usually at least about 5 wt. % of the first active compound. Some embodiments contain from about 25 wt. % to about 50 wt. % or from about 5 wt. % to about 75 wt. % of the first active compound.

### Methods of Use

This disclosure provides a method of treating cancer and blood disorders in a patient, comprising administering a therapeutically effective amount of a combination of a HMA and an XPO1 inhibitor, such as compound selected from Valtrate, Caffeic acid phenethyl ester (CAPE), and the pharmaceutically acceptable salts or hydrates of any of the foregoing. The disclosure provides additional methods of treating cancer and blood disorders in a patient, including methods of treating ovarian cancer and colon cancer, comprising administering a therapeutically effective amount of a combination of a HMA and an XPO1 inhibitor, such as a compound selected from Valtrate, Caffeic acid phenethyl ester (CAPE), and the pharmaceutically acceptable salts and hydrates of any of the foregoing to the patient.

The disclosure includes a method of treating a patient having a cancer or a blood disorder, the method comprising administering a therapeutically effective amount of a combination of a HMA and an XPO1 inhibitor, such as amount of Valtrate, Caffeic acid phenethyl ester (CAPE), or a derivative of either of the foregoing to the patient.

The XPO1 inhibitor and HMA combination may be administered by any method of pharmaceutical administration, including oral, topical, parenteral, intravenous, subcutaneous injection, intramuscular injection, inhalation or spray, sublingual, transdermal, intravenous, intrathecal, buccal, and rectal administration. In certain embodiments administration of Valtrate or Caffeic acid phenethyl ester (CAPE) is oral or parenteral.

Methods of treatment include providing certain dosage amounts of the first active compound to a patient. Dosage levels of either compound of the XPO1 inhibitor and HMA combination are from about 0.01 mg to about 140 mg per kilogram of body weight per day are useful in the treatment of the above-indicated conditions (about 0.5 mg to about 1 g per patient per day). In certain embodiments 0.1 mg to 5000 mg, 1 mg to 2000 mg, 1 mg to 1000 mg, 1 mg to 500 mg, 1 mg to 200 mg, 1 mg to 100 mg, 1 mg to 50 mg, 10 mg to 5000 mg, 10 mg to 2000 mg, 10 mg to 1000 mg, 10 mg to 500 mg 10 mg to 300 mg, 10 mg to 200 mg, 10 mg to 100 mg, 50 mg to 5000 mg, 50 mg to 2000 mg, 50 mg to 1000 mg, 50 mg to 500 mg, 50 mg to 200 mg, of one or both compounds of the XPO1 inhibitor and HMA combination are provided daily to a patient. In certain embodiments 0.1 mg to 5000 mg, 1 mg to 2000 mg, 1 mg to 1000 mg, 1 mg to 500 mg, 1 mg to 200 mg, 1 mg to 100 mg, 1 mg to 50 mg, 10 mg to 5000 mg, 10 mg to 2000 mg, 10 mg to 1000 mg, 10 mg to 500 mg 10 mg to 300 mg, 10 mg to 200 mg, 10 mg to 100 mg, 50 mg to 5000 mg, 50 mg to 2000 mg, 50 mg to 1000 mg, 50 mg to 500 mg, 50 mg to 200 mg per dose of one or both compounds of the XPO1 inhibitor and HMA combination are provided to the patient. In certain embodiments the dosage amount of Valtrate is 10 mg to 200 mg, 50 mg to 200 mg, or 50 to 150 mg per dose are provide to the patient, administered as 1 to 4 daily doses. In certain embodiments the dosage amount of CAPE is 10 mg to 200 mg, 50 mg to 200 mg, or 50 to 150 mg per dose are provide to the patient, administered as 1 to 4 daily doses. In certain embodiments the dosage of 5-AC is 100 to 500 mg, 300 mg, or 150 mg administered as 1 to 2 daily doses. In certain embodiments the dosage of tetrahydrouridine (THU) is 1-15 mg/kg, or about 50 to 2250 mg, 100 to 2000 mg, 200 to 1000 mg, 100 to 1000 mg, 200 to 1000 mg, or 200 to 600 mg administered as 1 to 2 daily doses.

Frequency of dosage may also vary depending on the particular disease treated. However, for treatment of breast cancer, a dosage regimen of 4 times daily or less is preferred, and a dosage regimen of 1 or 2 times daily is particularly preferred. Treatment regimens may also include administering the first active compound (Valtrate and Caffeic acid phenethyl ester) to the patient for a number of consecutive days, for example for at least 5, 7, 10, 15, 20, 25, 30, 40, 50, or 60 consecutive days. In certain embodiments the first active compound is administered for a period of 1 to 10 weeks and the amount and frequency of dosage is such that concentration of the compound in the patient's plasma in never less than 50% of the patient's plasma Cmax.

Treatment regimens may also include administering the first active compound to the patient for a number of days prior to cancer surgery (surgery to remove tumors including mastectomy and lumpectomy). For example, the first active compound may be administered to the patient for a number of consecutive days at 1 to 4 months prior to surgery. Treatment regimens may also include administering the first active compound to the patient in conjunction with radiation therapy, e.g., before, during or after radiation therapy.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disorder for the patient undergoing therapy.

Combination Therapy

The XPO1 inhibitor and HMA combination, may be used alone to treat breast cancer, including triple negative breast cancer, ovarian cancer, or colon cancer, or in combination with at least one additional active compound. Combination use includes an administering of the first active compound and additional active compound in a single dosage form, or in separate dosage forms either simultaneously or sequentially.

Suitable doses for the XPO1 inhibitor and HMA combination when used in combination with a second active agent are generally as described above. Doses and methods of administration of other therapeutic agents can be found, for example, in the manufacturer's instructions in the Physician's Desk Reference. In certain embodiments, the combination administration of XPO1 inhibitor and HMA combination with the additional active compound results in a reduction of the dosage of the additional active compound required to produce a therapeutic effect (i.e., a decrease in the minimum therapeutically effective amount). Thus, preferably, the dosage of an additional active compound in a combination or combination treatment method is less than the maximum dose advised by the manufacturer for administration of the additional active compound without combination administration of the first active compound. In certain embodiment this dosage is less than ¾, less than ½, less than ¼, or even less than 10% of the maximum dose advised by the manufacturer for the additional active compound when administered without combination administration of the first active compound.

The XPO1 inhibitor and HMA combination may be used to treat cancers and effect regression of tumors, including cancerous tumors. In certain embodiments, the patient is suffering from a cell proliferative disorder or disease. The cell proliferative disorder can be cancer, tumor (cancerous or benign), neoplasm, neovascularization, or melanoma. Cancers for treatment include both solid and disseminated cancers. Exemplary solid cancers (tumors) that may be treated by the methods provided herein include e.g. cancers of the lung, prostate, breast, liver, colon, breast, kidney, pancreas, brain, skin including malignant melanoma and Kaposi's sarcoma, testes or ovaries, carcinoma, sarcoma, and kidney cancer (renal cell). Cancers that may be treated with a XPO1 inhibitor and HMA combination also include bladder cancer, breast cancer, colon cancer, endometrial cancer, lung cancer, bronchial cancer, melanoma, Non-Hodgkin's lymphoma, cancer of the blood, pancreatic cancer, prostate cancer, thyroid cancer, brain or spinal cancer, and leukemia. Exemplary disseminated cancers include leukemias or lymphoma including Hodgkin's disease, multiple myeloma and mantle cell lymphoma (MCL), chronic lymphocytic leukemia (CLL), T-cell leukemia, multiple myeloma, and Burkitt's lymphoma. Particularly included herein are methods of treating cancer by providing XPO1 inhibitor and HMA combination to a patient wherein the cancer is a solid tumor or disseminated cancer.

Further included are methods of treating cancer by providing a XPO1 inhibitor and HMA combination to a patient wherein the cancer is selected from glioma (glioblastoma), acute myelogenous leukemia, acute myeloid leukemia, myelodysplastic/myeloproliferative neoplasms, sarcoma, chronic myelomonocytic leukemia, non-Hodgkin's lymphoma, astrocytoma, melanoma, non-small cell lung cancer, small cell lung cancer, cervical cancer, rectal cancer, ovarian cancer, cholangiocarcinoma, chondrosarcoma, or colon cancer.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy

A therapeutically effective amount of XPO1 inhibitor and HMA combination may be administered as the only active agents to treat or prevent diseases and conditions such as blood disorder diseases, undesired cell proliferation, cancer, and/or tumor growth or may be administered in combination with another active agent. A therapeutically effective amount of a XPO1 inhibitor and HMA combination may be administered in coordination with a regime of one or more other chemotherapeutic agents such as an antineoplastic drug, e.g., an alkylating agent, or a cytidine deaminase inhibitor, e.g. THU. In addition, other non-limiting examples of active therapeutics include biological agents, such as monoclonal antibodies or IgG chimeric molecules, that achieve their therapeutic effect by specifically binding to a receptor or ligand in a signal transduction pathway associated with cancer (e.g. therapeutic antibodies directed against CD20 (e.g. rituximab) or against VEGF (e.g. bevacizumab)).

Methods of treatment provided herein are also useful for treatment of mammals other than humans, including for veterinary applications such as to treat horses and livestock, e.g. cattle, sheep, cows, goats, swine and the like, and pets (companion animals) such as dogs and cats.

The disclosure provides pharmaceutical combinations (including compositions such as oral, injectable, or intravenous compositions) comprising (1) a hypomethylating agent (HMA) and (2) a compound selected from Valtrate or a derivative thereof and Caffeic acid phenethyl ester (CAPE) or a derivative thereof, and the pharmaceutically acceptable salts and hydrates of any of the foregoing to the patient. In certain embodiments the combination also includes a therapeutically effective amount of Tetrahydrouridine (THU). In certain embodiments the hypomethylating agent comprises 5-Azacytidine (5-AC), 5-aza-2′-deoxycitidine (DAC), or a combination thereof.

### EXAMPLES

### Example 1. The Nuclear Export Inhibitors Valtrate and CAPE Trigger Differentiation in vitro by Inhibiting XPO1, the Differentiation is Synergistically Magnified by the Addition of 5-AC

To treat AML HL 60 cell line, small molecule inhibitors of XPO-1 Valtrate and CAPE were used in an optimized low noncytotoxic dose, sufficient to cause an increase in lineage specific transcription factors within the nucleus that induce differentiation without apoptosis. The low dosages were purposed for use in combination with low dose 5-AC, Such as to position AML cells in a state of hypomethylated accessible chromatin with increased nuclear transcription factors. cell counts and morphologic alterations were observed. Dosages for this purpose were: Valtrate (6 μM), CAPE (4 μM), 5-AC (2.5 μM), and for comparing with Selinexor (20 nM) the same dosages were used for combinations. Combination of Valtrate or CAPE with 5-AC terminated proliferation while single agents did not (FIG. 3A, B) synergistic differentiation was maximal with Valtrate, 5-AC combination, big, rounded cells and spindle shaped cells observed indicated multiple terminal fate that could represent granulocytic and monocytic differentiation (FIG. 3, C). Spindle shaped cells were not observed with CAPE or Selinexor combinations with 5-AC or as single agents (FIG. 3B, C).

### Example 2. The XPO-1 Inhibitors, are Tested In Vivo as Single Agents in a Survival Experiment. 5-AC, and 5-AC in Combination with THU are Tested and Compared in the Same Patient-Derived Xenograft (PDX) Model with NPM1/FLT3 Mutations

Immunodeficient NSG mice are xenotransplanted with human NPM1/FLT3-mutated AML cells. Engraftment took 24 days to reach at least 40% and was established in 3 mice prior to treatment as confirmed by flow cytometry.

On day 24 mice were assigned for treatment groups (n=5 per group). The treatment groups are 1) Vehicle, 2) Valtrate 10 mg/kg by oral gavage, 3) CAPE 50 mg/kg by oral gavage, 4) selinexor 7 mg/kg by oral gavage, 5) 5-AC2 mg/kg subcutaneously, and 6) 5-AC(2 mg/kg) subcutaneously+THU (20 mg/kg) intraperitoneally. The addition of THU extends 5-AC half-life, by inhibiting the enzyme cytidine deaminase(CDA), which degrades 5-AC. Treatment started on day 24, 3×/wk. (FIG. 4A). Selected low dosages of Valtrate, CAPE, 5-AC and THU are adjusted for efficacy without toxicity while dose selection for selinexor was based on literature. Groups are compared with vehicle and with each other. To monitor progress periodic blood counts are analyzed along with distress signs. Distress signs were observed for euthanasia in accordance to institutional guidelines. Survival analysis shows benefits of Valtrate, CAPE, and selinexor compared to vehicle, however it is minimal (FIG. 4B) when compared to 5-AC group, which exhibited a survival benefit of more than 25 days, and 5-AC+THU, which had a survival benefit of more than 35 days, confirming the benefits of THU addition to 5-AC (FIG. 4B). Blood analysis confirmed the delayed onset of increased blast counts (WBCs), anemia as deceased (Hb), and thrombocytopenia as decreased platelets counts (FIG. 4C).

### Example 3. Efficacy of In Vivo Combinations of 5-AC/THU with the Nuclear Transport Inhibitors Valtrate and CAPE Compare to Selinexor in PDX Model—NPM1/FLT3 Mutation

A treatment challenge model of high tumor load was prepared using primary acute myelogenous leukemia (AML) patient cells with Nucleophosmin 1 and fins-like tyrosine kinase-3 mutations (NPM1/FLT3). Cells were injected into the tail vein of immunodeficient Nod-SCID-IL-2Rgamma-null mice (NSG, Jackson Laboratory) as 2×10⁶ cells IV (n=5 per group) at day 0 (D0). In order to reach a higher AML cell number in the bone marrow (tumor load) engraftment time was increased to 24 days. At D24≥40% bone engraftment was confirmed in 3 randomly selected mice. At D24 mice were divided randomly in to five groups (n=5 per group) and treatment started on the same day. Treatment groups were: 1) Vehicle, 2) 5-AC(2 mg/kg)/THU(20 mg/kg), 3) 5-AC(2 mg/kg)/THU(20 mg/kg)/selinexor (7 mg/kg), 4) 5-AC(2 mg/kg)/THU(20 mg/kg)/CAPE (50 mg/kg) and 5) 5-AC(2 mg/kg)/THU(20 mg/kg)/Valtrate(10 mg/kg). Treatment was then repeated three times a week. FIG. 5A illustrates the model and treatment plan. Efficacy between treatment groups and the vehicle group was compared. Vehicle mice were euthanized at distress in accordance with institutional guidelines. This occurred on average by day 35. In order to allow longer treatment time and improved analytic measurement discrimination between treatment groups euthanasia of all other treated groups was set at fixed time point on day 74. FIG. 5B. shows treatment plan and fixed time point euthanasia and D74.

To test for toxicity vs efficacy peripheral blood analysis provides an excellent tool. Pancytopenia is indicative of bone marrow suppression whereas stable counts without an increase in WBCs are indicative of efficacy without toxicity. Peripheral blood obtained by tail vein phlebotomy and serial blood count on day 0, day 35, and day 74 were measured by HemaVet blood lab. Mean±SD. P<0.01 significant. White blood cells count WBCs, hemoglobin Hb, and platelets counts displayed FIGS. 6. A,B,C. A sharp rise of WBCs counts for vehicle group on day 35 indicated high AML blast numbers while 5-AC, THU provided delay of distress and modest increase of WBCs day 74.

A WBCs count decrease on day 74 observed for the 5-AC, THU, and selinexor combination treatment group indicates a treatment limited benefit because it is associated with toxicity since the decrease is not only for AML blasts but also for normal healthy blood cells., This is confirmed by decreased hemoglobin (Hb) and platelets, indicating toxic pancytopenia FIGS. 6A, B, C. Both the 5-AC,THU, and CAPE group and 5-AC,THU, and Valtrate group were effective and non-cytotoxic as indicated by stable serial counts of WBCs, Hb, and platelets FIGS. 6A,B,C. For all groups femoral, tibial bones and spleens were removed. White bones indicate leukemia replacement has occurred, whereas red bones, which appear dark gray in FIG. 7A, top, indicate functional hematopoiesis. (Bone marrow myeloid content was evaluated by Giemsa staining bone marrow cells for evaluation (FIG. 7A, bottom). Flow cytometry detects and quantify human (hCD45) tumor load percentage in Median±IQR. p value Mann-Whitney test two-sided (FIG. 7B). Pale whitish bones and presence of blasts is present in high levels in vehicle group as confirmed by flow cytometry counts (FIGS. 7 A &B), and to much lesser degree in 5-AC,THU group. All (5-AC,THU, and selinexor), (5-AC,THU, and Valtrate) and (5-AC,THU, and CAPE) groups had lower than detectable AML cells in BM (FIGS. 7A &B).

Extramedullary tumor burden was evaluated in spleens by weight, photograph, and image analysis of histologic H&E stained sections. Spleens with infiltrating AML cells are recognized by being larger and homologous. Tumor burden was maximal in vehicle group. In the vehicle group the weight of spleen was >0.9 gram compared to about 0.020 grams in normal NSG mice. Further evidenced images and plotted cell counts of quantified image analysis of H&E stained sections (FIGS. 8 A,B,C). Unlike in BM, the 5-AC,THU, and selinexor combination group had additional extramedullary tumor load in spleens over the 5-AC and THU group, which could possibly be attributed to side effects to healthy splenic tissue (FIGS. 8 A,B,C). Normal spleen weight, size and histologic structure without AML cell infiltration, consistent with noncytotoxic combinational treatment is evident in groups of 5-AC,THU, and CAPE. and 5-AC,THU, and Valtrate. (FIGS. 8 A,B,C).

### Example 4. Survival Study Integrating High Tumor Load PDX Mouse Model of NPM1/FLT3 Mutations with Precision Medicine of Low, Non-Cytotoxic Combinational Dose of (5-AC/THU with Valtrate or CAPE) Compared to (5-AC/THU with Selinexor)

This example used the same model as example 3., AML with NPM1/FLT3 mutations cells are injected into tail vein of NSG mice, as 1×10⁶ cells IV (n=5 per group) at day 0 (D0). To challenge treatment efficacy, high AML tumor load engraftment≥45% reached on day 28, confirmed in 3 randomly selected mice. At D28 mice were divided to five groups (n=5 per group) and treatment is started on the same day. The fiver treatment groups are: 1) Vehicle, 2) 5-AC (2 mg/kg)/THU (20 mg/kg), 3) 5-aza (2 mg/kg)/THU (20 mg/kg)/selinexor (5 mg/kg), 4) 5-AC(2 mg/kg)/THU (20 mg/kg)/CAPE (50 mg/kg) and 5) 5-AC (2 mg/kg)/THU (20 mg/kg)/Valtrate (10 mg/kg). (FIG. 9A) Treatment was initiated on day 28, repeated three times a week and terminated on day 84 to observe survival.

The 5-AC,THU, and CAPE group and 5-AC,THU, and Valtrate combination group, had a no relapse and distress free period of total 200 days, and were euthanized for analysis. Survival for 116 day post treatment cessation is indicative of complete eradication of AML cells and permanent cure from the high 45% tumor load, and any possible AML cells sanctuary. This excellent efficacy is compared to the 5-AC,THU, and selinexor group that survived an average 140 days and the 5-AC,THU group that averaged 105 days, vehicle group euthanized on average day 46 at distress. Kaplan-Meier survival graph (FIG. 9C).

The curative effect of the 5-AC,THU, and combination CAPE and 5-AC,THU, and Valtrate combination is confirmed by bone marrow and peripheral blood analysis. Femoral and tibial bones were removed and photographed. White pale bones indicate leukemia replacement has occurred and is seen in three groups: 1) Vehicle, 2) 5-AC, and 3) THU. It is also seen in the 5-AC,THU, and selinexor combination at their distress points respectively. Red, dark bones, which appear dark gray in the figures, indicate functional hematopoiesis. This is observed only in relapse-free groups 5-AC,THU, and CAPE and 5-AC,THU, Valtrate. (FIG. 10A, bottom) Bone marrow cells from all groups, were analyzed by flow cytometry to determine human (hCD45) tumor load percentage. The 5-AC,THU, and CAPE and 5-AC,THU, Valtrate groups had no detectable hCD45, indicating complete remission (cure) as AML cells differentiation predictably terminates through the life span of a WBC. Other groups were analyzed at their respective distress points, and showed over 75% tumor load as they succumbed to AML burden despite prolonged distress free delay. (FIG. 8. A, top).

Furthermore, efficacy vs toxicity is best assessed by serial peripheral blood analysis of WBCs, hemoglobin Hb, and platelets. Five measures were taken on day 0, 45, 100, 140 and 200. Blood samples were analyzed by HemaVet blood lab. Mean±SD. P<0.01 significant.˜

Relapse free groups (5-AC,THU, and CAPE) and (5-AC,THU, and Valtrate) have successive stable successive values for WBCs, Hb and platelets reflecting nontoxic, high efficacy combinations. No increase in WBCs indicates no AML blasts pooling into blood. Increasing WBCs indicates AML blast presence (low efficacy treatment), while decreasing WBCs, Hb, or platelets is a sign of drug-induced toxic cytopenia. Importantly, in this model is a combination of increased WBCs (AML blasts) with decrease in Hb, and Platelets which reflect a tumor burden distress of bone marrow as seen in groups: Vehicle, (5-AC and THU), and (5-AC,THU, and selinexor) at their respective points of distress. (FIG. 10B.)

An additional valuable measurement is extramedullary splenic tumor burden, as evaluated by spleen weight and photograph. In spleen with high tumor burden the large AML cells are packed densely. Splenic tumor burden is evaluated by image analysis of histologic H&E stained sections. Tumor burden was maximal in vehicle, (5-AC and THU) and (5-AC,THU, and selinexor) groups at their respective points of distress with spleen weight >0.8 gram relative to only about 0.020 a gram average weight for normal NSG mouse spleen and the (5-AC,THU, and CAPE) and (5-AC,THU, and Valtrate) treated groups (FIG. 11B.) Similarly, in histologic cell count analysis of H&E stained sections (FIG. 11 A.) normal spleen weight, size and histologic structure without AML cell infiltration, consistent with noncytotoxic efficacious treatment is evident in groups of (5-AC,THU, and CAPE). and (5-AC,THU, and Valtrate). (FIGS. 11A,B,C).

### Example 5. Extending Combinational Treatments Efficacy to Additional Aggressive PDX Model of MLL Mutations in Fixed Time Point Analysis

AML leukemia with mixed-lineage (MLL)-mutated is aggressive. AML subtype model with the same experimental design and dosages discussed above was used (FIGS. 12 A, B) Bone marrow engraftment was confirmed at D7 rather than D24. At D35 mice were humanly euthanized in accordance with institutional guidelines as the vehicle group became moribund or lost 15% of their initial weight. Results are presented in (FIGS. 12A, B, C) and (FIGS. 13 A, B).

### REFERENCES

1. Grossmann, V., et al., A novel hierarchical prognostic model of AML solely based on molecular mutations. Blood, 2012. 120(15): p. 2963-72.
2. Rucker, F. G., et al., Chromothripsis is linked to TP53 alteration, cell cycle impairment, and dismal outcome in acute myeloid leukemia with complex karyotype. Haematologica, 2018. 103(1): p. e17-e20.
3. Fabbro, M. and B. R. Henderson, Regulation of tumor suppressors by nuclear-cytoplasmic shuttling. Exp Cell Res, 2003. 282(2): p. 59-69.
4. Welcker, M., et al., Nucleolar targeting of the fbw7 ubiquitin ligase by a pseudosubstrate and glycogen synthase kinase 3. Mol Cell Biol, 2011. 31(6): p. 1214-24.
5. Karin, M., et al., NF-kappaB in cancer: from innocent bystander to major culprit. Nat Rev Cancer, 2002. 2(4): p. 301-10.
6. Chahine, M. N. and G. N. Pierce, Therapeutic targeting of nuclear protein import in pathological cell conditions. Pharmacol Rev, 2009. 61(3): p. 358-72.
7. Thompson, M. E., BRCA1 16 years later: nuclear import and export processes. FEBS J, 2010. 277(15): p. 3072-8.
8. Fukuda, M., et al., CRM1 is responsible for intracellular transport mediated by the nuclear export signal. Nature, 1997. 390(6657): p. 308-11.
9. Fornerod, M., et al., CRM1 is an export receptor for leucine-rich nuclear export signals. Cell, 1997. 90(6): p. 1051-60.
10. Jardin, F., et al., Recurrent mutations of the exportin 1 gene (XPO1) and their impact on selective inhibitor of nuclear export compounds sensitivity in primary mediastinal B-cell lymphoma. Am J Hematol, 2016. 91(9): p. 923-30.
11. Gravina, G. L., et al., Nucleo-cytoplasmic transport as a therapeutic target of cancer. J Hematol Oncol, 2014. 7: p. 85.
12. Dover, G. J., et al., 5-Azacytidine increases HbF production and reduces anemia in sickle cell disease: dose-response analysis of subcutaneous and oral dosage regimens.

Blood, 1985. 66(3): p. 527-32.
13. Mao, L. and Y. Yang, Targeting the nuclear transport machinery by rational drug design. Curr Pharm Des, 2013. 19(12): p. 2318-25.

1. A method of treating cancer or a blood disorder in a patient, the method comprising administering a therapeutically effective amount of a combination comprising (1) a hypomethylating agent (HMA) and (2) a compound selected from Valtrate or a derivative thereof and Caffeic acid phenethyl ester (CAPE) or a derivative thereof, and the pharmaceutically acceptable salts and hydrates of any of the foregoing to the patient.

2. The method of claim 1, further comprising administering a therapeutically effective amount of Tetrahydrouridine (THU) to the patient.

3. The method of claim 2, wherein the hypomethylating agent comprises 5-Azacytidine (5-AC), 5-aza-2′-deoxycitidine (DAC), or a combination thereof.

4. The method of claim 1, wherein the patient is a patient having solid tumors and the therapeutically effective amount of the compound is amount sufficient to produce a decrease in the number and/or size of tumors in the patient.

5. The method of claim 1, where the patient is a patient having a blood disorder or blood cancer in which the patient has either aberrant blood cells or an abnormal blood cell count and a therapeutically effective amount is an amount sufficient to produce a decrease in the number of aberrant blood cells in the patient's blood relative to the number before the patient was administered the combination of claim 1 or improve the patient's blood cell count relative to the patient's blood cell count before the patient was administered the combination of claim 1.

6. The method of claim 1, wherein the therapeutically effect amount of each of the hypomethylating agent (HMA) and the compound selected from Valtrate or a derivative thereof and Caffeic acid phenethyl ester (CAPE) or a derivative thereof is a total daily dose of 0.01 milligrams per kilogram (mg/kg) to 100 milligrams per kilogram patient weight.

7. The method of claim 6, wherein the therapeutically effect amount of each of the hypomethylating agent (HMA) and the compound selected from Valtrate or a derivative thereof and Caffeic acid phenethyl ester (CAPE) or a derivative thereof is a total daily dose of 0.02 milligrams per kilogram to 50 milligrams per kilogram patient weight.

8. The method of claim 6, wherein the daily dose is administered for at least 3, 5, 7, 10, 20, or 30 consecutive days.

9. The method of claim 1, wherein the patient has cancer and the cancer is leukemia, myelodysplastic syndrome (MDS), or lymphoma.

10. The method of claim 9, wherein the cancer is leukemia.

11. The method of claim 1, wherein the patient has cancer and the cancer has a Nucleophosmin 1 and fins-like tyrosine kinase-3 gene (NPM1/FLT3) mutation or a Mixed-lineage leukemia (MLL) mutation.

12. The method of claim 1, wherein an additional active compound is administered and the additional active compound is an anthracycline class drug, a taxane class drug, an antimetabolite, an alkylating agent, a platinum agent, or a vinca alkaloid.

13. The method of claim 13, wherein an additional active compound is administered and the additional active compound is daunorubicin, doxorubicin, epirubicin, idarubicin, valrubicin, docetaxel, paclitaxel, abraxane, taxotere, 5-fluorouracil, 6-mercaptopurine, capecitabine, cytarabine, gemcitabine, mechlorethamine, cyclophosphamide, chlorambucil, melphalan, ifosfamide, cisplatin, carboplatin, oxaliplatin, nedaplatin, vinblastine, vincristine, vindesine, vinorelbine, vincaminol, vineridine, or vinburnine.

14. The method of claim 1, wherein the hypomethylating agent is administered for a period of 1 to 10 weeks and the amount and frequency of dosage of the hypomethylating agent is such that concentration of the hypomethylating agent in the patient's plasma is never less than 50% of the patient's plasma Cₘₐₓ for the hypomethylating agent.

15. The method of claim 1, wherein the patient is a human.

16. A method of treating acute myeloid leukemia (AML) in a patient comprising administering a therapeutically effective amount of a combination of
(1) a hypomethylating agent (HMA) and (2) an XPO1 inhibitor
to the patient, wherein the patient is identified as having leukemia with either a MLL mutation or one or both of an NPM1 mutation and an FLT3 mutation.

17. The method claim 16, wherein the XPO1 inhibitor is Valtrate or a pharmaceutically acceptable salt or hydrate thereof or Caffeic acid phenyl ester or a pharmaceutically acceptable salt or hydrate thereof.

18. A method of treating acute myeloid leukemia in a patient comprising determining that the patient has leukemia with either a MLL mutation or one or both of an NPM1 mutation and an FLT3 mutation and administering an effective amount of a combination of (1) a hypomethylating agent (HMA), (2) an XPO1 inhibitor, and optionally THU to the patient.

19. The method of claim 18 wherein the XPO1 inhibitor comprises valtrate or a pharmaceutically acceptable salt or hydrate thereof or Caffeic acid phenyl ester or a pharmaceutically acceptable salt or hydrate thereof and the HMA comprises 5-Azacytidine (5-AC) or a pharmaceutically acceptable salt or hydrate thereof, 5-aza-2′-deoxycitidine (DAC) pharmaceutically acceptable salt or hydrate thereof.
